(19) United States
(12) Patent Application Publication
(10) Pub. No.: US 2019/0350672 A1
(43) Pub. Date: Nov. 21, 2019

(54) METHOD AND DEVICE FOR ENHANCED TRANSDERMAL VISUALIZATION OF MEDICAL DEVICES

(71) Applicant: Visionary Medical Therapies, Inc., Irmo, SC (US)

(72) Inventors: Christopher K. SMITH, Irmo, SC (US); John F. BLACK, San Mateo, CA (US); Gilbert S. CLARKE, Steamboat Springs, CO (US)

(21) Appl. No.: 16/138,960

(22) Filed: Sep. 21, 2018

Related U.S. Application Data
(60) Provisional application No. 62/673,079, filed on May 17, 2018.

(51) Int. Cl. A61B 90/00; A61M 1/36; A61B 5/00; A61L 31/08; A61L 31/06; A61L 31/04

(52) U.S. Cl. CPC A61B 90/39; A61M 1/3655; A61B 5/0062; A61B 5/0071; A61L 31/08; A61L 2420/06; A61L 31/048; A61B 2090/3933; A61B 2090/3945; A61B 2090/3941; A61L 2420/08; A61L 31/06

(57) ABSTRACT

Implantable and/or insertable devices having a near-IR fluorescing material that allows the device to be visualized through a patient's skin. Also described herein are apparatuses for imaging devices having a near-IR fluorescing material, and methods of imaging devices having a near-IR fluorescing material from within the body, including methods of modifying an implanted device having near-IR fluorescing material.

### CROSS REFERENCE TO RELATED APPLICATIONS

This patent application claims priority to U.S. Provisional Patent Application No. 62/673,079, filed on May 17, 2018 (titled “METHOD AND DEVICE FOR ENHANCED TRANSDERMAL VISUALIZATION OF MEDICAL DEVICES”), which is herein incorporated by reference in its entirety.

### INCORPORATION BY REFERENCE

All publications and patent applications mentioned in this specification are herein incorporated by reference in their entirety to the same extent as if each individual publication or patent application was specifically and individually indicated to be incorporated by reference.

### FIELD

The present invention relates to improved visualization of implantable/insertable medical devices, including grafts, such as venous implants. In particular described herein are methods and apparatuses for transdermal (through the skin) visualization of medical devices.

### BACKGROUND

It would be very helpful to be able to visualize medical implants, such as grafts, needles, shunts, and the like, through the skin. For example, vascular implants, such as arteriovenous grafts, which must be accessed after insertion or implantation, may be difficult to locate and orient through the patient's skin. The most commonly performed hemodialysis access operation is a subcutaneous placement of an arteriovenous graft, which is made from a biocompatible tube. The biocompatible tube can be made of, for instance, a fluoropolymer such as polytetrafluoroethylene. One end of the tube is connected to an artery while the other end is connected to a vein. The arteriovenous graft is typically placed either in the leg or arm of a patient. An arteriovenous graft may be used to provide hemodialysis, a process whereby the patient's blood is filtered and toxins are removed using an extracorporeal dialysis machine.

These types of grafts may have a number of problems in their construction and as such a number of different approaches to graft design have been developed. In U.S. Pat. No. 7,144,381 issued Dec. 5, 2006 to Gertner there is a hemodialysis system and method described with adjustable members. In U.S. Pat. No. 7,147,617 to Henderson et al. and issued Dec. 12, 2006 there is another example of an arterio-venous shunt graft. In U.S. patent application 2006/0229548 published Oct. 12, 2006 there is an arteriovenous graft system with access valve systems along with methods of using them.

While the use of these arteriovenous grafts substantially improves the hemodialysis process it is clear that venous grafts have a set of problems associated with their use. Access into the graft is typically accomplished by nursing staff or medical technicians without the training to read ultrasound or other techniques for finding the graft to access with a needle. Accordingly, it is typical that these technicians and staff use either touch or a previously done “diagram” to place the needle. Because of the locations and the depth of the grafts these personnel are almost always attempting to access the graft blindly. Stick site errors result in graft damage, shortening the usable lifetime, and have the patient presenting with complications such as pseudoaneurysms, aneurysms, thrombus, clots and blockage with the long-term possibility of total occlusion of the graft and the need for replacement. This is not to mention the potential pain and discomfort the patient experiences. The resulting complications cost tens of millions of dollars in invasive treatments to remedy these problems. That doesn't include lost work time and the problems associated with further surgical intervention. There are relatively few implantation sites in the body, so premature graft failure can cause a significant shortening of life expectancy relative to the probability of finding a donor match.

Similarly, a vascular access port is a vascular implantable port that is designed for subdermal implantation. It is designed for repeated access to the vasculature, for example, for administration of a desired product by injection. Typically, a vascular access port is fixed in position by suturing to underlying fascia in the desired location. Both single and dual access ports (or multiple devices) are frequently utilized on a patient. While the use of these ports substantially improves the repeated access problems to the vasculature, it is clear that ports have problems associated with their use. Injection into the port is typically accomplished by nursing staff or medical technicians without the training to read ultrasound or other techniques for finding the port to access with a needle. Accordingly, it is typical that these technicians and staff thus use either touch or a previously done “diagram” to place the needle. Because of the location and the like of the port these personnel are almost always attempting to access the port blindly. Stick site errors may result in port damage, needle damage (loss of sharpness), and in the patient presenting complications such as pseudoaneurysms, aneurysms, thrombus, clots and blockage with the possibility of total occlusion of the port needing replacement. This is not to mention the potential pain and discomfort the patient experiences.

Accordingly, it would be useful if there were additional methods, guidance techniques, more visible grafts or the like that would aid the healthcare worker in identifying and accessing implanted or inserted medical devices, such as vascular grafts and ports, through the patient's skin.

### SUMMARY OF THE DISCLOSURE

Described herein are methods and apparatuses for the use of selectively emitting (e.g., electromagnetic emitting, radioluminescing and/or photoluminescing materials as part of a vascular medical device or implant (graft, stent, access port, etc.). In particular, the use of materials that emit electromagnetic radiation, radioluminscence and or photoluminescence over specific regions and in particular patterns may provide safer and easier to use implants and devices.

Specifically, described herein are methods and apparatuses (e.g., systems, devices, etc.) for using one or more infrared (and particularly near-IR) markers to visualize an implant beneath the skin. Also described herein are apparatuses for detecting the near-IR marker(s) as well as tools (e.g., needles, forceps, sutures, etc.) marked with the same or a different near-IR marker.

Although previous work (e.g., US 2010-0010339 and US 2010-0198079, herein incorporated by reference in its entirety) described the use of ultraviolet (UV) light (e.g., <400 nm) and visible fluorescing materials as part of a vascular graft and/or port, in practice these devices have proven difficult to use. In addition to the possible danger of exposing living tissues to UV light, the penetration depth of UV is through tissue is typically quite shallow, preventing identification of the implant beyond a few hundred microns of implantation depth. The method and apparatuses described here represent a substantial improvement over this earlier work. In particular the methods and apparatuses (devices and systems) described herein may be used with a material that emits one or more (and in particular, patterns of) materials that fluoresce in the near infra-red wavelength range following excitation by deep red or near-IR light. The use of near-IR light may have numerous advantages, particularly compared to certain wavelengths of blue and UV light which may damage some tissues of the body (e.g., due to photosensitization), and ultimately lead to perivascular damage and tissue “burns” from photochemical damage (phototoxicity).

In general, the materials described herein may be referred to as near-IR luminescent materials that emit light (also typically within the near-IR range) upon stimulation by a different (e.g., shorter) frequency of near-IR light. In some variations the near-IR marker(s) will emit a near-IR wavelength that is longer than the near-IR wavelength used for illuminating them to luminescence. However, so-called up-conversion materials that can absorb at long wavelength (e.g., near IR) and emit at (near-IR and/or visible) short wavelengths may also be used. These materials typically need to be charged, or prepped to trigger emission of a visible photon when an IR one is absorbed. In some variations, these materials may fluoresce in the visible upon illumination with an IR photon, and thus the imaging system may be configured to visualize them. The near-IR luminescent materials described herein may include near-IR fluorophores that may be excited with shorter wavelengths than they fluoresce with (e.g., they fluoresce at long wavelengths). In some variations a chemiluminescent material (e.g., luciferase, etc.) emitting in the SWIR may be used.

For example, described herein are methods and apparatuses for using multiple near-IR fluorescing materials (near-IR absorbing and emitting materials) in a distinctive pattern that can be observed when an appropriate near-IR energy (e.g., electromagnetic energy) source is used to illuminate the material, including through tissue.

In general, the near-IR coatings may be excited and visualized when inserted or implanted into the tissue to a depth of greater than at least 5 mm (e.g., greater than at least 7.5 mm, greater than at least 10 mm, greater than at least 12 mm, greater than at least 13 mm, greater than at least 14 mm, greater than at least 15 mm, etc.). Thus, the maximum depth may be a depth of about 5 mm or greater (e.g., about 7.5 mm, about 10 mm, about 12 mm, about 13 mm, about 14 mm, about 15 mm, about 17 mm, etc.). The near-IR frequency for absorption and emission by the near-IR coating(s) may be between, for example, 650 nm and 1300 nm (e.g., the absorption may be between 650-800 nm and the emission may be between, 800-1200 nm). In some variations the near-IR absorption and emission may be within the 700-1300 nm range, or within the 800-1300 nm range.

In some variations, the methods and apparatuses described herein may be configured to monitor the dissolution rate of a biodegradeable (e.g., a biodegradable implant, such as a sinus implant) by incorporating a fluorophore into the biodegradable material forming the implant, and monitoring fluorescence intensity over time. As the material biodegrades, the florescence (e.g., the near-IR fluorescing material) signal may vary. The user of near-IR may be preferable because it may not degrade or damage the material as much as other wavelengths. Similarly, a near-IR fluorescent material may be incorporated into a drug-releasing layer (coating, etc.), and may monitor the release of drug over time. For example, if a product includes disposable/degradable (e.g., biodegradeable) material having different drugs that may be released at/over different times, the different near-IR florescent materials in the different layers could indicate which drug is being released at any given time.

Any appropriate implantable or insertable device may be marked and/or coated with the near-IR material. For example, a graft (e.g., a vascular graft) may be marked with (or may incorporate) a near-IR fluorescing material. A vascular graft can be made easier to use by including a near-IR fluorescing composition that can be visualized through the skin (or other tissues). Examples of implantable or insertable medical devices that may include a near-IR fluorescing material may include (but are not limited to) devices that are inserted under the skin (transcutaneous), and/or devices inserted into a body cavity, and/or devices inserted into the vasculature, device anchored or inserted into a bone, and the like. For example, implantable or insertable medical devices that may include a near-IR fluorescing material may include (but are not limited to): vascular grafts, bone screws, artificial joints (e.g., artificial hips, knees, etc.), cardiac pacemakers, neural pacemakers/neural stimulators, breast implants, artificial disks, spinal rods/screws, intrauterine devices (IUDs), stents, coronary stents, ear tubes, artificial eye lenses, implantable cardiac defibrillators, etc.

For example, the implantable or insertable medical device that may include a near-IR fluorescing material may be a sensory or neurological implant, such as intraocular lens, intrastromal corneal ring segment, cochlear implant, tympanostomy tube, and neurostimulator. An implantable or insertable medical device that may include a near-IR fluorescing material may be a cardiovascular implant, such as artificial heart, artificial heart valve, implantable cardioverter-defibrillator, cardiac pacemaker, and coronary stent. An implantable or insertable medical device that may include a near-IR fluorescing material may be an orthopedic device, such as pins, rods, screws, and plates used to anchor fractured bones while they heal. An implantable or insertable medical device that may include a near-IR fluorescing material may be a contraceptive implant, such as hormone-releasing device(s) and intrauterine devices. An implantable or insertable medical device that may include a near-IR fluorescing material may be a drug-delivery device. An implantable or insertable medical device that may include a near-IR fluorescing material may be a cosmetic device (including prosthesis), such as breast implant, nose prosthesis, ocular prosthesis, and injectable filler. Other devices that may include a near-IR fluorescing material may be surgical materials, such as suture material, surgical mesh, clips (including staples), and the like.

In some variations, surgical material, such as sponges, bandages, gauzes, surgical packing materials, etc., may have a near-IR fluorescent material (e.g., fluorophore) incorporated on them, which may help prevent them from being left behind in a patient following a surgical procedure. For example, the surgical material incorporating a near-IR fluorescing material may be used during procedure, and at the end of the procedure (or during the procedure) near-IR light may be used to monitor and/or check the patient to confirm that surgical material was not unintentionally left behind in the patient.

In some variations, the tools used to access an implanted or implantable device may also or alternatively include a near-IR fluorescing material, which may aid in visualization when processing (implanting, removing, modifying, etc.) an implant. For example, surgical tools, such as needles, scissors, forceps, retractors, probes, etc. The amount, intensity or type (e.g., emission wavelength) may be different on any of these devices that include a near-IR fluorescing material. For example, materials that are to be implanted may be configured to fluoresce in the near-IR wavelength when excited by the appropriate excitation near-IR wavelength more strongly than surgical tools that may access them through the tissue. For example, in some variations, the tools may be made to fluoresce differently (e.g., at different wavelengths) than an implant. Similarly, as mentioned above, the surgical materials may be made to fluoresce at a different wavelength(s). Thus, the detection may be determined by a yes/no indication at a particular wavelength, which may be automated (e.g., automatically detected), rather than requiring manual detection, even after a near-IR fluorescing implant has been implanted.

In general, the near-IR fluorescing material on the apparatus may be incorporated on an outer surface of the implanted or insertable device(s) in any appropriate manner. For example, the near-IR material may be a paint, dye, coatings, impregnation, micelles, supported on nano-particles, etc. or any other near-IR material may be used. The near-IR material may be arranged in a pattern, design, and/or may include symbols, including alphanumeric text, that may help orient a medical practitioner when visualizing them. Two or more near-IR fluorescing materials may be used. In particular, two or more materials that emit in different near-IR wavelengths may be used (which may be excited by the same or different wavelengths), and/or two or more near-IR fluorescing materials that emit at nearly the same wavelength, but that are excited at different wavelengths may be used, and/or two or more near-IR fluorescing materials that both emit and are excited at different wavelengths may be used.

By including a near-IR fluorescing material in the implant or insertable device, and exposing it to the appropriate near-IR excitation wavelength (or a range of wavelengths including the appropriate near-IR excitation wavelength), a healthcare worker attempting to access the implanted or insertable device within the tissue will be able to visualize the device. For example, if a graft includes a near-IR marker as described herein, the healthcare worker (using a near-IR imaging device as described herein) may be able to see the device fluorescing under the skin.

For example, described herein are exemplary grafts (e.g., arteriovenous grafts) comprising a near-IR fluorescing material, wherein the near-IR fluorescing material is positioned such that upon exposure to an energy source (e.g., a source of near-IR radiation emitted in the range in which the near-IR fluorescing material absorbs) the graft or a portion of the graft fluoresces in the near-IR sufficiently to improve the visibility of the graft by a health care worker attempting to access the graft, who may view the field of view with a imaging apparatus configured to allow imaging in the near-IR range that the near-IR fluorescing material on the device fluoresces (emits).

Thus, also described herein are apparatuses that detect the near-IR emission of the implantable or insertable medical devices comprising a near-IR fluorescing material. These devices may be referred to as near-IR imaging devices, and may be configured to include an output (e.g., display, screen, etc.) for visualizing the near-IR fluorescing material. Any of these devices may also be configured to include one or more emitters for emitting in the excitation wavelength in the near-IR that is specific to the near-IR fluorescing material on the devices being imaged. Any of these apparatuses may also include one or more near-IR excitation light sources. Any of these devices may also include one or more near-IR filters, for filtering out near-IR wavelengths from the received light that is not the near-IR wavelength emitted by the target near-IR fluorescing material on the device (e.g., the implant or insertable device). These devices may include a silicon-based near-IR sensor (e.g. CCD). Any of these devices may also be configured to receive and display visible light as well, and may display near-IR separately and/or as an overlay onto the visible light display.

For example, any of these apparatuses may image near-IR and full-color images and may illuminate an area under observation with continuous or discrete near-IR light; in some variations the apparatus may also illuminate in blue/green light and with red light. For example, the methods and apparatuses described herein may interlace RGB and the near-IR imaging to alternately show the skin and then the subdermal graft. This could be used, for example, with skin fiducial markers (e.g., that may be exogenously applied, for example, by placing dots using a surgical pen) to guide the user (e.g., when making a needle stick into the implant). Thus, in some variations the apparatus may include two or more sensors (e.g., two or more CCDs) or one sensor (e.g., one CCD) with an alternating filter, to allow visible or near-IR light through. The near-IR and visible light may be displayed concurrently or separately (e.g., alternating, e.g., one of the red light and near-IR light may be switched on and off periodically). Light (including in particular the near-IR light) returning from the area under observation may be directed to one or more sensors which may be configured to separately detect the visible light (e.g., blue light, the green light, and the red or the combined red light/near-IR light) and the near-IR light. The red light spectral component and the near-IR light spectral component may be determined separately, in synchronism with the switching of the light(s). Thus, any of these apparatuses may include a light source providing near-IR and (optionally) visible light to an area under observation, a camera having one or more image sensors configured to detect near-IR and in some variations separately detect visible light returned from the area under observation, and a controller in signal communication with the light source and the camera. The controller may be configured to control the light source to continuously or non-continually (e.g., pulsed) illuminate the area under observation with the near-IR light (and optionally visible light). Further, any of these system and apparatuses may include multiple near-IR emitters, including emitters that emit at different near-IR wavelengths that may be used to separately excite different near-IR materials marking the device to be visualized. Thus, the various light sources may be switched on and off periodically in synchronism with the acquisition of the near-IR images in the camera. By arranging the LEDs spatially the methods and apparatuses may build up stereoscopic info on the target to guide to the correct depth as well as the best site on the skin surface to stick.

In some variations, the controller may be configured to determine from sensor signals representing near-IR light the near-IR light spectral component. The imaging system may display receiving image signals corresponding to the visible light, and the separately or in combination the one or more near-IR spectral component and rendering therefrom a full-color or colorized display showing the near-IR light (e.g., as a visible color). This display may include the visible light image of the area under observation. The display may show the separately determined near-IR light spectral component and may render therefrom an image of the near-IR emission in the area under observation. In some variations, exogenous contrast may be added to the blood stream, e.g., in the form of an ICG solution, and the patency of the graft may be gauged,

Any of these apparatuses may be configured as video imaging systems and may record, display and/or transmit the images. Display may be in real time. The display may be presented to a screen near (above, adjacent, behind, etc.) the patient. In some variations, the display may be a projection onto a surface and/or directly onto the patient. For example, the output (e.g., display) may project a color (e.g., visible light color) on a region corresponding to the near-IR emission on the patient's body.

The image sensors may employ an interlaced scan or a progressive scan. For example, a line scan CCD and mosaicking may be used to build an image up and/or an edge detect and correlation function may be used to build the image.

In any of these apparatuses, the light source may include an illuminator emitting a substantially constant intensity of visible light and/or near-IR light over a continuous spectral range, and a plurality of movable filters disposed between the illuminator and the area under observation for transmitting temporally continuous near-IR light and temporally discontinuous visible light. In some variations, the light source may include an illuminator emitting a substantially constant intensity of near-IR light (and optionally visible light) over a continuous spectral range, and may include one or more first dichroics for separating the visible light and the near-IR light (or multiple near-IR wavelengths), one or more shutters for transforming the separated light into temporally discontinuous near-IR light wavelengths (and/or visible light), and in some variations a second dichroic for combining the temporally discontinuous light for transmission to the area under observation.

In some variations, the light source includes a first illuminator emitting a substantially constant intensity and/or switched near-IR excitation light.

Disclosed herein are methods for visualizing an implant (e.g., an arteriovenous graft) within a patient's body, the method comprising: applying a near-IR illumination to the skin of a patient in an excitation wavelength, illuminating, though the skin, an implant comprising a near-IR absorbing and emitting material, emitting, from the implant, a near-IR emission, detecting in an sensor outside of the patient's body, the near-IR emission and displaying the near-IR emission on a visible display.

Any of these methods may include implanting the implant within the body, as well as modifying the implant after implantation (e.g., contacting with a needle or other medical instrument). Any of these methods may also include accessing the implant through the tissue once identified (e.g., for thrombectomy, debris removal, recovery of a semi-permanent implant, modification, etc.).

As mentioned above, the implant may be any appropriate implant, including a vascular access port or other medical device that can also be made easier to use by near-IR fluorescence, as described herein. For example, described herein are vascular access ports, at least a portion of which comprises a biocompatible near-IR fluorescing material. Any of these devices (e.g., implants) may include more than one near-IR fluorescing material that have different emitting and/or excitation properties and may therefore be differently visualized. For example, in some variations, the different near-IR fluorescing materials may be arranged in a different overlapping or non-overlapping patterns on the device/implant (e.g., adjacent each other, etc.). In some variations, upon exposure to the near-IR excitation energy, the device (e.g., port or a portion of the port) subintimally implanted may fluoresces in the near-IR sufficiently to improve the visibility of the location of the graft to a health care worker attempting to access the port using the near-IR visualizing apparatuses described herein.

For example, described herein are arteriovenous shunt (AV shunt) implant devices that may include: an elongated tubular (e.g., AV shunt) body, the body having an inner lumen formed of an inner layer; a first middle layer extending at least partially over the inner layer, the first middle layer comprising a substrate and a near-infrared (near-IR) dye that absorbs and emits in the near IR wavelength range; and a first outer layer extending over the first middle layer and sealing the first middle layer between the first outer layer and the inner layer. In some variations the near-IR dye is at a concentration of between 0.001% to 0.5% w/w.

Any appropriate near-IR dye may be used, including (but not limited to): 1,1′,3,3,3′,3′-Hexamethylindotricarbocyanine iodide. The near-IR dye may be at a concentration of between 0.001% w/w and 0.1% w/w.

The tubular body may comprise a second middle layer separate from the first middle layer, wherein the second middle layer comprises a second substrate and a second near-IR dye. The second middle layer may be covered by the first outer layer or a second outer layer extending over the second middle layer and sealing the second middle layer between the second outer layer and the inner layer. The second near-IR dye may be the same as the first near-IR dye or a different near-IR dye, and the second substrate may be the same material as the first substrate or a different material. For example, the substrate may be silicone. The inner layer may be, for example, polytetrafluoroethylene.

The first outer layer may comprise a biocompatible material that is greater than 50% transparent to light between about 700-850 nm (e.g., greater than 55%, greater than 60%, greater than 65%, greater than 70%, greater than 75%, greater than 80%, greater than 85%, greater than 95%, etc.). In some variations the first outer layer comprises polytetrafluoroethylene.

The near-IR dye may be in a pattern over the inner layer, or it may be uniformly distributed. For example, the near-IR dye may be in a pattern that is striped (e.g., helical, ringed, etc.), a checkerboard pattern, etc.

For example, an arteriovenous shunt (AV shunt) implant device may include: an elongated tubular body the body having an inner lumen formed of an inner layer; an arterial region comprising a first middle layer surrounding and extending partially along a first length of the inner layer, the first middle layer comprising a first substrate and a first near-infrared (near-IR) dye that absorbs and emits in the near IR wavelength range; and a first outer layer extending over the first middle layer and sealing the first middle layer between the first outer layer and the inner layer; and a venous region comprising a second middle layer surrounding and extending partially along a second length of the inner layer, the second middle layer comprising a second substrate and a second near-infrared (near-IR) dye that absorbs and emits in the near IR wavelength range; and wherein the second middle layer is covered by the first outer layer or a second outer layer. In some variations the first and second near-IR dyes are at a concentration of between 0.001% to 0.5% w/w. The first near-IR dye and the second near-IR dye may be the same (e.g., both may be 1,1′,3,3,3′,3′-Hexamethylindotricarbocyanine iodide) or they may be different.

Also described herein are methods for guiding hemodialysis. For example, a method may include: applying a near-IR illumination to the skin of a patient in an excitation wavelength; detecting, in a sensor outside of the patient's body, near-IR emission from an arteriovenous shunt (AV shunt) within the patient's body, wherein AV shunt comprises a near-IR absorbing and emitting material; and guiding insertion of one or more needles through the patient's skin into the AV shut using the detected near-IR emission.

Guiding may include displaying an image indicating the near-IR emission on a visible display. The visible display may be a screen, a projection, and/or a virtual reality or augmented reality display. For example, guiding may comprise displaying one or more needle insertion sites on an image on the patient's body or on a representation of the patient's body. Applying may comprise applying from a hand-held near-IR imaging reader. Detecting may comprise detecting from a hands-free near-IR imaging reader.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIG. 1A is a schematic overview of one method of visualizing a device having a near-IR fluorescing material implanted or inserted into a patient's body.

FIG. 1B is a schematic illustration of one example of a system for visualizing a device having a near-IR fluorescing material within a patient's body.

FIG. 1C is a schematic illustration of an example of a system for visualizing a device having a near-IR fluorescing material within a patient's body.

FIG. 2A is a perspective view of an embodiment of a device having a near-IR fluorescing material arranged in two bands of near-IR fluorescing compound on an outer surface of the device. These bands may be different near-IR fluorescing materials, e.g., materials having different emission and/or excitation wavelengths.

FIG. 2B is a perspective view of an embodiment of the present invention where there is a graft which is entirely made with a near-IR fluorescing material.

FIG. 3 is a perspective view showing multiple stick sites.

FIG. 4 is a perspective view of a graft showing different near-IR fluorescing materials indicating front wall and back wall of the graft.

FIG. 5 is a perspective view of an embodiment of the present invention where there are two bands of near-IR fluorescing materials.

FIG. 6 is a perspective view of an embodiment of the present invention where there is a port with a ring entirely made with a single near-IR fluorescing material.

FIG. 7 is a perspective view of a graft showing a pattern of differently near-IR fluorescing materials extending down the length of the graft.

FIG. 8 is a perspective view of another example of a graft having a pattern of differently near-IR fluorescing materials.

FIG. 9 is a schematic illustration of one example of a hand-held near-IR imaging device.

FIGS. 10A and 10B illustrate examples of alternative configuration for emitters (LEDs) and sensors (CCDs) for a near-IR imaging device.

FIGS. 11A-11B show one example of a portion of an arterial venous shunt (AV shunt or fistula), one example of a device having a near-IR fluorescing material as described herein.

FIG. 11C is an example of an arterial venous shunt in which the arterial side and the venous sides are labeled with near-IR fluorescing material. The curving region between the arterial and venous sides of the shunt is unlabeled (or may be differently labeled. The labeling may be enclosed or encapsulated.

FIG. 11D is a longitudinal cross-section through a region of a graft (e.g., AV shunt) labeled with a near-IR dye material.

FIGS. 12A-12E illustrate alternative locations and examples of AV shunts.

FIG. 12F shows regions (e.g., arterial and venous regions) that are labeled in the exemplary AV shunts shown in FIGS. 12A-12E.

FIGS. 13A-13C illustrate a first example of a method of identifying an implant (e.g., AV shunt) within a patient through the skin using a near-IR fluorescing material as described herein. In FIG. 13A, the shunt is shown implanted within the patient, between an artery (e.g., brachial artery) and a vein (e.g., antecubital vein). FIG. 13B illustrates illumination of the patient's arm using one or more near-IR emitting LEDs. FIG. 13C illustrates excitation, through the skin, of the near-IR emitting colorant on the arterial portion of the shunt and the venous portion of the shunt.

FIGS. 13D-13E illustrate visualization, e.g., through the skin, of the AV shunt shown in FIGS. 13A-13C. FIG. 13D shows an image of the patient's arm (not visible) illuminating the near-IR dye marked AV shunt; the image may be displayed (e.g., on a monitor, etc.) unprocessed or minimally processed, as shown. In FIG. 13D, the arterial and venous portions are separately marked, while the bent/curved region between them is not marked. In FIG. 13E the image of the near-IR emitting/excited markings is shown following processing (which may be done in real time) to detect a continuous edge of the implant. The edge of the implant is shown as a dashed line overlaying the ‘real’ image. FIG. 13F shows an image similar to that shown in FIG. 13E, with markings (e.g., cross-hairs, targets, etc.) showing where to insert the needle(s).

FIGS. 13G-13I illustrate projections of the display on the patient's arm (e.g., by back projecting or by augmented reality). FIG. 13G illustrates the projection of an image, detected in real time using near-IR imaging, as described herein, to illuminate the arterial and venous arms of the AV shunt implanted in the arm; the image may be minimally processed (e.g., showing the emitted regions). FIG. 13H is an example in which the real-time near-IR image has been processed, e.g., to detect edges from the near-IR excited portion detected, and the processed image of the AV graft projected onto the patients arm. FIG. 13I also shows a processed image of the AV graft projected onto the subject's arm, showing both the edges of the underlying graft, as well as recommended targets for needle puncture.

FIGS. 14A-14C illustrate examples of tracking of needle punctures for an implanted AV stent using a near-IR imaging system as described herein. In FIG. 14A, a real-time or near-real time near-IR image of an AV stent labeled with a near-IR marker (e.g., dye) is shown. The image may be processed to display proposed/suggested needle puncture regions, shown by cross-hairs. In FIG. 14B, a processed of the near-IR marked AV stent may also include markings showing prior needle puncture regions. FIG. 14C illustrates a processed near-IR marked AV stent illustrating both historical (past) needle markings as well as a set of proposed or suggested puncture regions.

FIG. 15 illustrates one example of a display showing a processed near-IR image of an AV stent that has been marked with a near-IR marker as described.

FIG. 16A is an example of a system for detecting near-IR marked implants, such as AV stents, in real time, beneath a subject's skin.

FIG. 16B is an example of a system for detecting near-IR marked implants, such as AV stents, in real time, beneath a subject's skin. FIG. 16B may include a projector for projecting images onto the patient's skin, and/or an augmented reality output.

FIG. 17A is an example of a calibration image for a near-IR imaging system.

FIG. 17B is an example of a near-IR image taken through the skin after removal of an implant coated with a near-IR emitting material that was not encapsulated (e.g., showing near-IR emitting residue. The top implant was coated with 0.1% w/w of HITCI (e.g., 1,1′,3,3,3′,3′-Hexamethylindotricarbocyanine iodide)

FIG. 17C is an example of a near-IR image of a graft implanted beneath the tissue; the graft has been coated with 0.015% HITCI.

FIG. 17D is an example of a near-IR image of two grafts implanted 2-4 mm beneath the skin. The upper implant was coated with 0.1% w/w of HITCI and covered with a protective outer covering. The lower implant was coated with 0.015% HITCI and covered with a protective outer covering. Near-IR excitation was provided by LEDs emitting at 750 nm (30 nm bandwidth); the camera recording images passed >800 nm. In FIG. 17D, the image was automatically scaled by the camera to the maximum intensity pixel.

### DETAILED DESCRIPTION

Described herein are implantable and/or insertable devices having a near-IR fluorescing material that allows the device to be visualized through a patient's tissue, including through the patient's skin. Also described herein are apparatuses for imaging devices having a near-IR fluorescing material, and methods of imaging devices having a near-IR fluorescing material from within the body, including methods of modifying an implanted device having near-IR fluorescing material. In some variations, the devices having a near-IR fluorescing material may include distinctive and/or informative patterns of near-IR fluorescing material on the outside of the device. For example, described herein are vascular devices and implants including a near-IR fluorescing material. The devices having a near-IR fluorescing materials and patterns may be particularly well adapted so that they may be easily visualized thorough tissue (e.g., greater than 5 mm, 6 mm, 7 mm, 8 mm, 9 mm, 10 mm, 11 mm, 12 mm, 13 mm, 14 mm, 15 mm, etc., of tissue) without damage to the tissue or the eyes of the physician or patient.

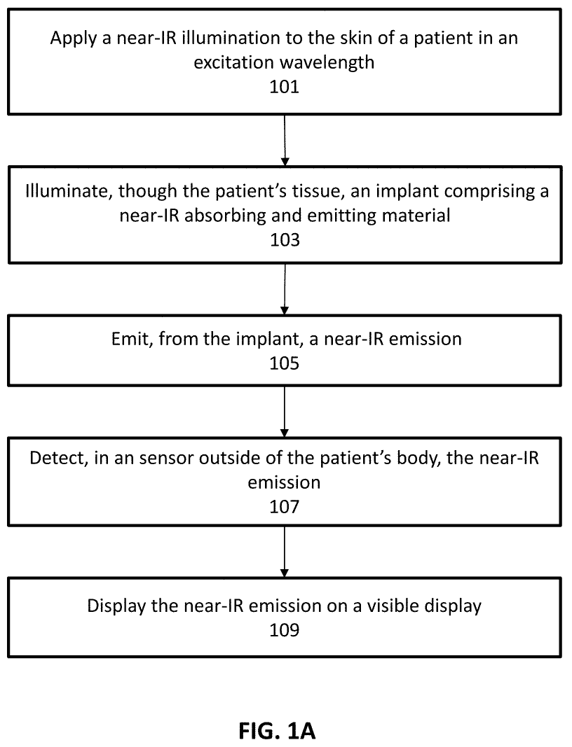

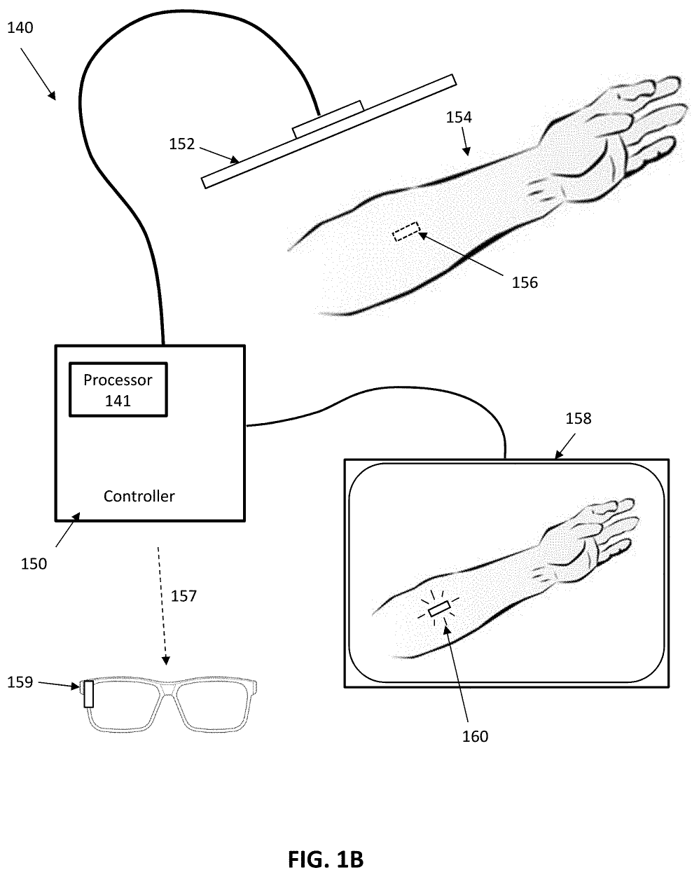

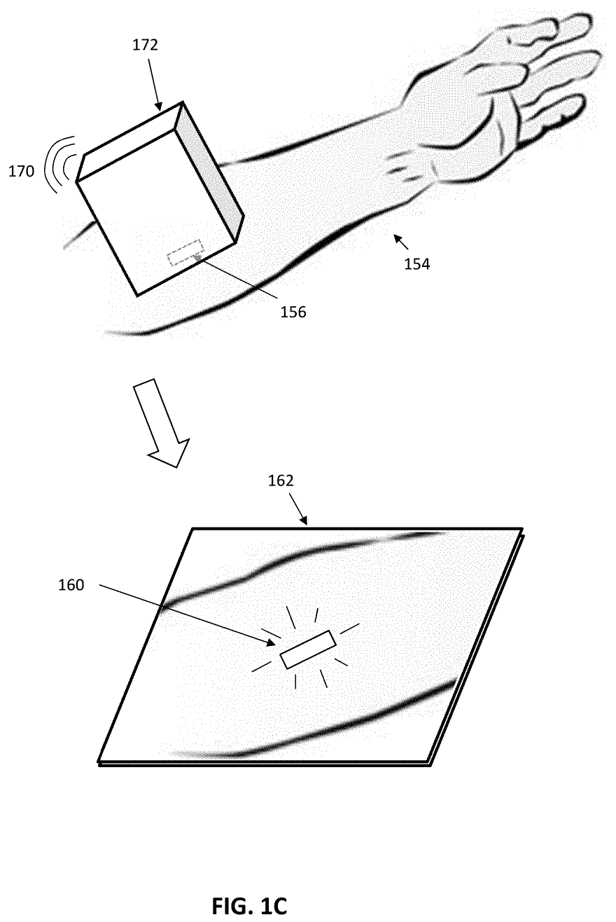

FIG. 1A shows an example of an exemplary method of visualizing an implant within a patient's body, including visualizing the implant through the overlaying tissue (e.g., skin, fat, muscle, etc.). For example, the method may include applying a near-IR illumination to the skin of a patient in an excitation wavelength 101. The near-IR illumination may be applied by an emitter of an imaging system that also includes a receiver with one or more sensors for receiving a near-IR wavelength. The emitter typically emits a long wave red wavelength for example from 650-700 nm, or a near-IR wavelength (e.g., between 700-1300 nm) and may include a near-IR LED (e.g., in some variations, one or more different wavelengths may be used). The method may also include illuminating, though the patient's tissue (e.g., skin), an implant comprising a near-IR absorbing and emitting material 103. The method may further include emitting, from the implant, a near-IR emission 105. The implant having a near-IR fluorescing material typically emits the near-IR emission in response to the absorption of near-IR energy in the absorption wavelength range of the near-IR fluorescing material in/on the implant. The near-IR emission may be sensed by detecting in a sensor outside of the patient's body 107. Thereafter, the near-IR emission may be displayed 109, e.g., on a visible display.

In general, in any of these methods and apparatuses described herein, the near-IR fluorescing device is visible using near-IR imaging apparatuses such as, but not limited to, those described herein and shown schematically in FIGS. 1B-1C. Any of these methods may also generate a map, e.g., a map of the subcutaneous position and/or orientation of the implant relative to the body. In some variations the near-IR fluorescing device is configured to absorb short-wave infra-red wavelengths (e.g., absorbing in the range of 703 nm-790 nm and emitting in the range of, e.g., 800-875 nm). The near-IR fluorescing material in the device may be uniformly distributed through the device, or it may be arranged in a pattern, including a readable pattern.

In general, the near-IR fluorescing devices described herein do not generate a significant amount of reactive oxygen species (ROS) or other significant photodynamic therapeutic effects, which may otherwise cause perivascular damage, alter the blood flow, etc. In some variations the near-IR colorant in the graft has a (fluorescence) quantum yield of >0.5. The colorant in the near-IR fluorescing device may be configured so as to avoid eluting/leaching. Further, in some variations, the near-IR fluorescing material in the device may be encapsulated and/or surrounded by a dense material, such as a polymer, that isolates it from ingress or diffusion of oxygen.

The near-IR fluorescing devices described herein may be visible to a depth of at least 5 mm under the surface of the tissue, e.g., to a depth of a least about 7.5 mm in depth (e.g. 7.5 mm to 1 cm). The near-IR fluorescing devices may be visible through all skin pigment types at this depth, and different skin pigments may be transparent in the near-IR wavelengths used. In some variations, the concentration of near-IR fluorescing material (e.g., dye, colorant, etc.) in the device and/or the thickness of the near-IR fluorescing material may be configured to produce >75% probability of absorption of an impinging excitation photon. The peak absorption coefficient of the colorant in the excitation wavelength range for a near-IR fluorescing material may be greater than about 150,000 l/mol*cm. The fluorescence quantum yield of the near-IR fluorescing material may be greater than 0.5 for a wavelength in the excitation range.

In general, the near-IR fluorescing materials described herein (used in the near-IR fluorescing devices) may be stable, and its optical properties may not degrade significantly over a 12-month implantation time. For example, the colorant may be photostable and thermostable. Its optical properties (e.g., absorption coefficient, peak emission intensity or fluorescence quantum yield, etc.) may be stable for more than 6 months, more than 9 months, more than 1 year, etc.

In general, the near-IR fluorescing materials described herein are biocompatible and may be sterilized (e.g., exposed to sterilizing heat and/or sterilizing treatments such as EtO gas sterilization) without degrading.

Any appropriate near-IR fluorescing compound may be integrated as a coating, mixture or polymer and used as part of a near-IR fluorescing device as described herein. For example, the near-IR fluorescing compound (or composition) may include a near-IR fluorophore (or fluorochrome or chromophore) may be, for example, one or more of: 2,9-Di(tridec-7-yl)-anthra[2,1,9-def:6,5,10-d′e′f′]diisoquinoline-1,3,8,10-tetrone (eg., N,N′-Bis(1-hexylheptyl)-perylene-3,4:9,10-bis-(dicarboximide)); Per-fluoro version of Perylene (e.g., N,N″-Bis(2,2,3,3,4,4,4-heptafluorobutyl)-3,4,9,10-perylene dicarboximide; Langhals 5b Quaterrylene Biscarbox Diimide; HepPTC, N,N′-Diheptyl-3,4,9,10-perylenedicarboximide (2,9-Diheptylanthra[2,1,9-def:6,5,10-d′e′f]diisoquinoline-1,3,8,10(2H,9H)tetrone); N,N′-Bis(1-hexylheptyl)-perylene-3,4:9,10-bis-(dicarboximide); Indocyanine (IR 125 FoxGreen CardioGreen); HITC (2-[7-(1,3-dihydro-1,3,3-trimethyl-2H-indol-2-ylidene)-1,3,5-heptatrienyl]-1,3,3-trimethyl-3H-indolium iodide or perchlorate; Hexacyanin 3); and DTTC IODIDE (3-ethyl-2-[7-(3-ethyl-2(3H)-benzothiazolylidene)-1,3,5-heptatrienyl]-benzothiazolium iodide).

In particular, the implants described herein may include a near-IR dye such as HITCI (e.g., 1,1′,3,3,3′,3′-Hexamethylindotricarbocyanine iodide). The near-IR dye may be used at a concentration of about 0.001% to about 0.5% w/w (e.g., about 0.002% w/w to about 0.15%, w/w, about 0.005% w/w to about 0.1% w/w, etc.). Outside of these ranges (e.g., outside of 0.005% to about 0.1% w/w of HITCI) the material may not emit appreciably when excitation near-IR light is applied (above 0.5% w/w or in some variations above 0.25% w/w, etc.) the dye may quench, while below about 0.001% w/w the excitation may not be great enough. Preliminary results with comparable amounts of DTTCI and ICG did not show appreciable near-IR florescence when used with the polymeric implants described herein. The rylene family of dyes are also likely to work in approximately the same concentrating range as HITCI.

Any of the near-IR dyes described herein may be coated, or combined with a substrate (e.g., a polymeric substrate, such as silicone) and/or encapsulated with a substrate, and applied to the implant.

Also described herein are methods of providing intelligible near-IR fluorescing markings on an implant that include: applying, onto a surface of an implant, a marking medium. The marking medium typically includes a near-IR dye (e.g., a liquid or a viscous substance), so that the marking medium is highly absorptive of radiation in the near infrared range of at least about 750 nanometers in wavelength, and fluoresces in response to radiation excitation in the near infrared range to produce fluorescent radiation of wavelengths longer than the wavelength of the excitation. The near-IR dye may be an organic dye that is poorly absorptive of radiation in the visible spectral range (e.g., about 400 to 700 nanometers) so that the markings are substantially transparent to light in the visible spectral range. The dye may be highly absorptive of radiation in the range of about 750 to 900 nanometers, and the fluorescent radiation may be produced principally in the range of about 800 to 1100 nanometers. For example, in some variations the near-IR dye is HITCI. In some variations the dye may be IR-125.

The marking medium may be applied to the implant in any appropriate manner (e.g., spray coating, dipping, etc.). In some variations the marking medium is applied by jet printing. The dye may be present in a concentration of about 0.005 to 0.05 percent by weight of the medium (e.g., between 0.001% w/w to about 0.5% w/w, between about 0.005% to about 0.05% w/w, etc.).

### Imaging Apparatus

In general, apparatuses for visualizing a near-IR fluorescing devices described herein may both emit and receive near-IR light. In some variations, the emitter(s) may be LED emitters or a laser excitation source. In some variations the emitter is one or more LEDs that are configured to emit light in the excitation near-IR wavelength range of the near-IR fluorescing devices. Any of these apparatuses may include a detector (near-IR detector) that is configured to receive and detect near-IR energy emitted by the near-IR fluorescing devices. For example, the detection apparatus may include a silicon-based camera (detector) that is sensitive to the emission wavelengths of the near-IR fluorescing devices. Alternatively, in some variations, the detector comprises a GaAs or InGaAs array detector (e.g., particularly when using near-IR fluorescent materials that emit at between, for example, 1000-1300 nm).

The visualizing apparatuses may include one or more outputs (e.g., screens, monitors, projectors, etc.) that may display an image of the near-IR fluorescence detected. The near-IR image may be converted to a visible light image that may be pseudo colored and/or intensity-mapped, and may be displayed alone, or overlaid onto a visible light image (e.g., of the patient's body, etc.).

In any of these apparatuses, the apparatus may be configured for use under normal room (e.g., hospital) lighting. This may be achieved, e.g., by using near-IR sensors that do not react to visible light, and/or by filtering near-IR light outside of the emitted range (e.g., between about 800-1200 nm). In some variations, the apparatus may create a constant tissue imaging plane/distance ameliorating the need for complex autofocusing mechanisms. In some variations, the apparatus may be used against the skin which may be referred to as contact imaging; contact imaging may also reduce or eliminate the effect of ambient (e.g., room) light.

FIGS. 1B and 1C illustrate generic variations of imaging apparatuses (e.g., systems) that may be used to visualized, and/or help implant, adjust, modify, and/or remove a near-IR fluorescent device. In FIG. 1B, the apparatus includes a system 140 that includes a near-IR emitter (e.g., laser, LED(s), etc.) and one or more near-IR sensors (e.g., near-IR sensitive cameras, CCDs, etc.) arranged as part of an imaging reader 152. The imaging reader 152 in this example may be positioned apart or over the patient, e.g., the patient's body 154 (e.g., arm, leg, torso, etc.) in order to image a near-IR fluorescent device 156 that is shown already implanted into the body in this example. In some variations the near-IR imaging reader may be a hand-held device. Alternatively or additionally, the near-IR imaging reader may be configured to be mounted to a holder (e.g., a stand, a boom arm, a mount, etc.). In some variations, the near-IR imaging reader may be configured to be hands-free. The near-IR imaging reader may be pressed against the tissue (e.g., skin) or it may be separated from the tissue. The imaging reader may include one or more optical filters (e.g., for filtering non-IR light and/or light outside of the absorption/emission range for the near-IR fluorescent material on the device.

The near-IR imaging reader may be connected (wirelessly or via a wired connection) to a controller 150. The controller may include the hardware, software and/or firmware for controlling the near-IR imaging apparatus, including the imaging reader, the filters, the near-IR sensors, the near-IR light sources (emitter), any visible light camera and/or visible light source (not shown), wireless communications, and/or outputs (e.g., displays, projectors, screens, etc.). In FIG. 1B, the system also includes a display or monitor 158 connected (via a wired, as shown, or wireless connection). The display may show just the near-IR images 160, showing the device fluorescing (after converting to a visible image); in some variations, the display may also display (e.g., concurrently, as an overlay, etc.) the visible light image, as shown. One or more outputs may be provided. In this example, an additional or alternative display may be a wearable display, such as glasses 159 or lenses that may receive images from the controller 150 for display to a person wearing them. Thus, any of these apparatuses may be configured as augmented reality displays, showing the near-IR fluorescent device overlaid in real time with the patient.

FIG. 1C shows another example of an imaging apparatus. In this example, the apparatus is configured as a hand-held imaging reader that may include the controller, near-IR light source(s), e.g., LEDs, near-IR sensor(s), e.g., CCD(s), any optics (e.g., filters, etc.) and wireless communication circuitry for communicating with one or more outputs 162 (displays, such as a screen/monitor, projector, wearable display, etc.). In this example, the hand-held imaging reader 172 includes a lens at the distal end that may be held against or near the tissue (e.g., patient's arm 154) in order to image a near-IR fluorescent device 156 by emitting a near-IR light in the absorption range of the near-IR fluorescent device, and detecting the near-IR emission of the near-IR fluorescent device 160, so that this image may be displayed. In some variations, the near-IR imaging reader may be configured to be used with a mount or holder, including an adjustable holder that may allow hands-free operation of the apparatus. For example, the apparatus may be used with a mount, stand, arm (e.g., boom-arm), or the like.

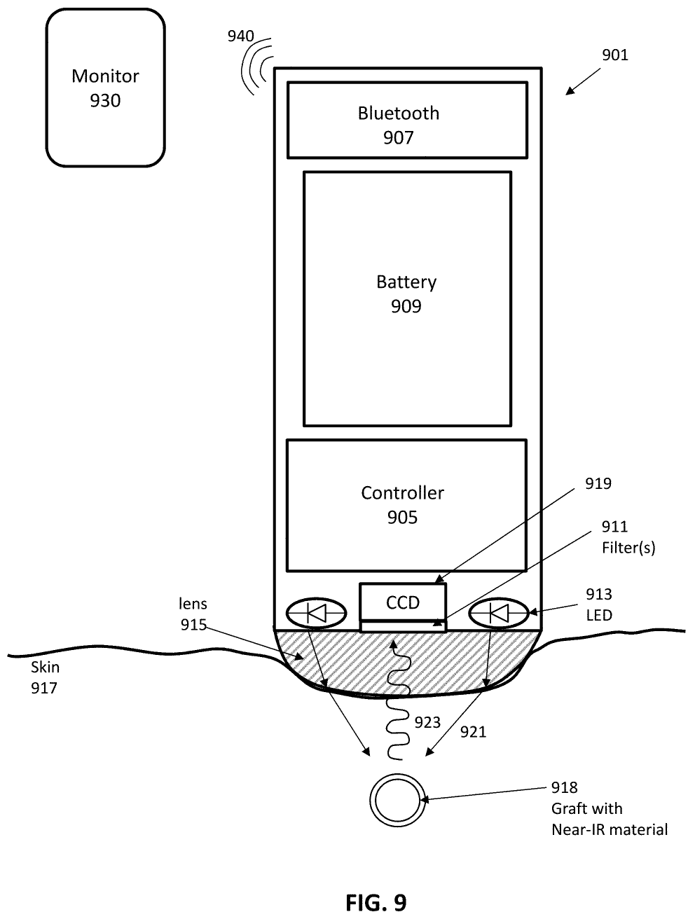

FIG. 9 illustrates an example of an imaging system that includes a hand-held imaging reader 901. The reader may be palm-sized (or larger) and may be held adjacent or against the tissue to provide imaging of any implanted/inserted device that fluoresces in near-IR. In some variations, the apparatus may include a slight curvature on a distal window surface that may displace some skin components between the window and the implant (e.g., graft), shortening the distance over which the excitation and emission photons have to travel. Thus, the imaging end of the device may be held or pressed against the tissue 917, as shown in FIG. 9. The imaging reader may be moved across the skin to located and/or image near-IR fluorescent device 918 (shown as a graft) within the tissue.

In FIG. 9, the hand-held reader includes the imaging sensor for detecting near-IR light in the wavelength(s) emitted by the near-IR fluorescent device. The imaging sensor in this example is a near-IR sensitive CCD 919. The reader may also include one or more near-IR light sources (emitters), shown in this example as a plurality of near-IR LEDs 913. The device may also include one or more filters 911 over the sensor(s) and/or over the near-IR emitters. One or more lenses 915 may also be used. In FIG. 9, the lens is a sapphire lens, which may advantageously be biocompatible, easy to clean, and ‘hard’ (e.g., difficult to damage). Multiple lenses, emitters, sensors, filters, and/or other optical components may be used. Any of these elements may be controlled by the controller 905. In the example shown in FIG. 9, the controller may include hardware, software and/or firmware for imaging a near-IR fluorescent device. The controller may include control circuitry such as one or more processors, one or more clocks, one or more data stores (e.g., memory), one or more power control circuits, one or more wireless communication circuits. In FIG. 9, the reader includes Bluetooth circuitry 907 configured to allow it to communicate with one or more displays (e.g., monitors 930, wearable devices, etc.). The controller may also control the power, such as controlling the power from the battery 909, and/or recharging or discharging the battery. The output may be a pad (e.g., iPad).

In operation, the apparatus may be moved around and may display (in real time) the scanned output of any near-IR emission from the body. The hand-held imaging reader 901 may continuously or periodically emit 921 near-IR light at the wavelength(s) that are absorbed and cause emission 923 from the near-IR fluorescent device.

In variations in which different near-IR absorbing/emitting materials are included as part of the near-IR fluorescent device, the apparatus may be configured to distinguish between the different near-IR absorbing/emitting materials. For example, the apparatus may multiple filters that may differentiate between different emitted frequencies of near-IR light from the near-IR fluorescent device. Alternatively or additionally, the reader may differentially control the emitter (near-IR light source) from emitting at different near-IR frequencies that are absorbed by the near-IR fluorescent device. The controller may, for example, switch between detection and/or illumination of different near-IR frequencies and may distinguish between the different frequencies so that they may be differently displayed. For example, emission and detection may be time-locked.

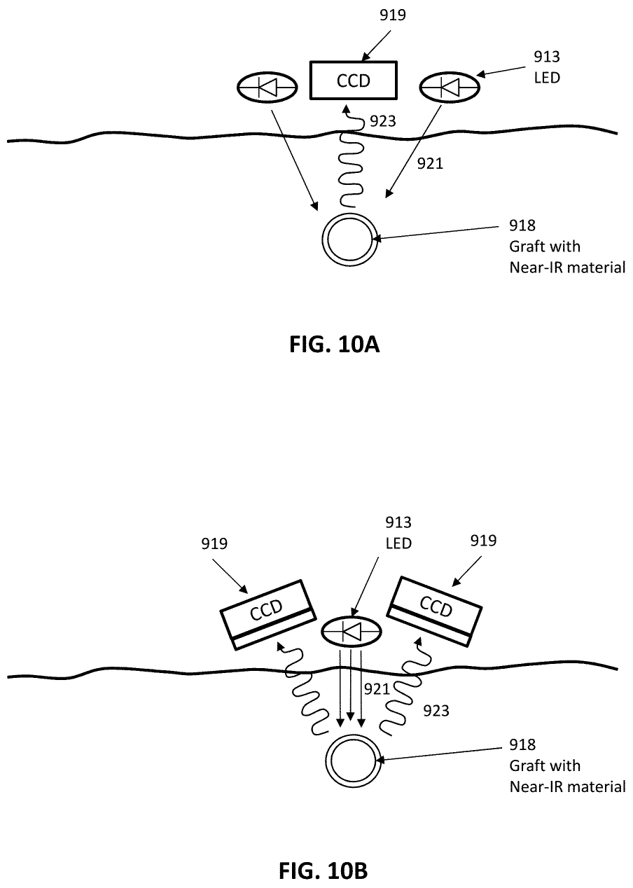

FIGS. 10A and 10B illustrate different variations of arrangements of emitters (near-IR light sources) and sensors (near-IR detectors) that may be used. In FIG. 10A, similar to the configuration shown in FIG. 9, the near-IR light sources 913 are arranged peripherally around one or more near-IR sensors 919. The near-IR sensor may be insensitive to the emitted near-IR wavelength(s) and/or it may include one or more filters and/or barriers to block detection of the emitted near-IR light (not shown). The emitted light 921 penetrates into the tissue and at least some is absorbed by the near-IR fluorescent device 918, causing it to emit near-IR in a different wavelength 923. This near-IR light emitted by the near-IR fluorescent device may take a relatively short path back to the sensor 919 where it can be detected, amplified, filtered, etc. and an image generated for display to the user.

In FIG. 10B, the relative positions of the near-IR light source(s) 913 and sensors 919 are reversed, so that the light emitted by the imaging reader may more directly illuminate the near-IR fluorescent device; the offset positions of the multiple different near-IR sensors 919 may be used to provide stereo images, for example, and/or may provide a larger imaging field.

Any of these apparatuses may be configured to receive structured fluorescence (e.g., emitted near-IR light from spatially distinct dye locations in the near-IR fluorescent device). For example, a graft having multiple different near-IR fluorescing materials may be used to monitor the patency of the device over time using, e.g., ICG fluorescence.

In some variations, the apparatus may also include guidance structures (e.g., channels, guides, etc.) that may also be displayed graphically to allow the user to access an imaged near-IR fluorescent device. For example, in some variations a hand-held imaging reader may include a needle carrier on an outer housing of the reader to help guide cannulation (e.g., of a near-IR fluorescent device configured as a port or graft). The display may include visual cues (e.g., cross-hairs, guides, etc.).

Alternatively or additionally, the surgical tool (e.g., needle, probe, suture, etc.) to be used in conjunction with the near-IR fluorescent device may include a near-IR fluorescing material (which may be different or the same as the near-IR fluorescing material of the near-IR fluorescent device). This may also aid in visualizing. For example, a visualizable needle, using the same near-IR wavelengths as the graft material may be used so that the user may see both the needle and the near-IR fluorescent device (e.g., graft).

For example, a device for locating a fluorescent implant within a patient's body may include: an outer enclosure or housing that may be water-tight (and/or sterilizable, including “wipe-down” or chemically sterilizable), and may include a distal portion and a proximal portion. The device may further include a near-IR transparent, biocompatible and/or sterilizable window or lens on one end (e.g., the distal end) of the housing. The device may further include one or more near-IR emitting sources (e.g., LEDs) that are behind or within the window/lens. The near-IR sources may be within the housing. The device may also include one or more near-IR sensors, such as a CCD sensor array behind or within the window/lens. As mentioned, any of these devices may include an optical filter that may be between the window/lens and the sensor. Further, any of these devices may include one or more power sources, such as batteries, within the housing. Finally, the device may include a controller for controlling operation of the device. The controller may include or control power control circuitry and/or communication circuitry, and/or imaging circuitry. One or more inputs, such as a button, dial, slider, trigger, switch, etc., may be in communication with the controller. In some variations the input may be on the housing; alternatively or additionally, the input may be remote and/or wirelessly communicating with the device. Thus, in some variations, the device includes a wireless transceiver within the housing (and in some variations as part of the controller).

The near-IR light source(s) may be activated on command to provide an excitation source for the fluorophore component of the near-IR fluorescent device. The sensor (e.g., CCD) may detect the fluorescence photons emitted by the fluorophore from the near-IR fluorescent device following excitation. Radiation in the correct wavelength range may be detected by the device, which may include a long pass filter interposed between the CCD and window.

Any of these devices may form an image or images (e.g., in real time) of the near-IR fluorescent device based on fluorescence intensity received.

As mentioned, any of these devices may include a guide (e.g., a guide path along or through the housing), which may help the user to insert a tool (e.g., standard cannulation needle) and to position the tool at the correct angle of insertion and/or at the correct point on the skin so as to ensure that the tool will contact the near-IR fluorescent device correctly. For example, where the tool is a needle and the implanted near-IR fluorescent device is a graft, the guide on the near-IR imaging device may help the needle enter the lumen of the graft and not graze the side wall or miss the graft entirely.

In any of these devices, the window may be on the distal portion of the device and may have a slight positive curvature (convex surface) to ensure positive contact between the imaging unit and the tissue (e.g., skin). The slight positive curvature may also be used to maintain a constant distance between the skin surface and the sensor plane (e.g., may avoids the need for an autofocusing mechanism). In some variations, the slight pressure on the hand piece may causes the positive curvature surface to press into the skin, slightly displacing tissue between the imaging hand piece window and the near-IR fluorescent device, thereby reducing the distance through which excitation and emission photons have to traverse, which may help increase the sensitivity of the device, allowing near-IR fluorescent device to be detected at greater depths than might otherwise be possible.

In any of these reader or detection devices described herein, the light sources (e.g., the one or more excitation LEDs) may be spatially offset from the sensor(s) (e.g., CCDs) so that by alternately firing different light sources, the apparatus may build up spatially registered information about the fluorescence (e.g., providing depth information). Alternatively or additionally, multiple sensors may be used and offset slightly, as shown in FIG. 10B. The apparatus may interrogate one or more sensors (e.g., CCDs) in a temporal sequence to build up spatially registered information about the origin of the fluorescence (e.g., depth information).

Any of these apparatuses may be used to establish a baseline flow and patency for an implanted near-IR fluorescent device, such as a vascular graft. For example, a method of use may include implanting a near-IR fluorescent device such as a graft that fluoresces in the near-IR range. The implanted near-IR fluorescent device may be imaged with blood flowing through the lumen of the graft. A fluorescent contrast agent may be perfused into the graft while imaging (e.g., in the presence of contrast agent). Images from before and during perfusion may be subtracted (e.g., subtracting contrast-enhanced images from a no-contrast image) to form an image of the blood flow in the vessel.

In any of the devices and methods described, the near-IR images of the near-IR fluorescent device may be stored. For example, images of the near-IR fluorescent device may be stored in the patient's electronic health record. In some variations, these images may be provided with one or more landmarks (e.g., physiological, external marks) that may provide a map for later access to the implant. The patient may be given a copy (electronic or paper) of the near-IR fluorescent device image post-implantation. This image may be provided to a health-care provided to assist in accessing the near-IR fluorescent device. For example, these images may be provided to a dialysis clinic at time of dialysis, even if the later health-care provider does not have a near-IR imaging apparatus available.

### Examples

FIGS. 1-6 illustrate examples of near-IR fluorescing materials. Any of these examples may be adapted as described herein to include a single near-IR fluorescing material, or multiple different near-IR fluorescing materials that may be arranged in distinct patterns. For example, described herein are devices including at least a portion that is fluoresces in the near-IR. For example, described herein are grafts, such as AV grafts, that including a near-IR fluorescing material which fluoresces upon application of near-IR energy in a particular absorption/excitation wavelength range. The fluorescing region can include just the injection sites of the graft, or the entire device, which can be totally or partially under the skin during use. This may help overcome the limitations and problems of the prior art for those medical technicians attempting to insert a needle into an AV graft, for example.

As used herein an arteriovenous graft is a biocompatible tube which is subcutaneously placed for access by a healthcare worker during hemodialysis. One end of the tube is connected to an artery while the other end is connected to a vein. Typically the insertion of the AV graft is by placement in the leg or arm of a patient. The biocompatible tube can be made of, for instance, a fluoropolymer such as polytetrafluoroethylene.

Blood flows from the artery, through the graft and into the vein. To connect the patient to a dialysis machine, two large-bore hypodermic needles are inserted through the skin and into the graft. Blood is removed from the patient through one needle, circulated through the dialysis machine, and returned to the patient through the second needle. This process is often performed for over four hours a day, three times a week. It is clear that insertion of the needle through the skin and into the graft should be as accurate as possible each time because of the problems associated with poor needle insertion as described above.

The term biocompatible near-IR fluorescing material may relate to a material which can be incorporated in, coated on or used to make an implantable or insertable device, such as an AV graft. These compositions may fluoresce with an intensity that may be directly proportional to the intensity of the near-IR light source, i.e. the more intense the near-IR light, the more intense the resulting near-IR emission will be.

One method of producing the near-IR fluorescing device is to incorporate a near-IR sensitive compound directly into the polymer matrix. The polymer can be injection molded or the like directly into the graft shape from there. Examples of plastic which could incorporate the near-IR absorbing/emitting compound may include polyol(allyl carbonate)-monomers, polyacrylated, polyethylenes, polypropylenes, polyvinyl chloride, polymethylmethacrylates, cellulose acetate, cellulose triacetate, cellulose acetate propionate, cellulose acetate butyrate, polyacetal resins, acetyl cellulose, poly vinyl acetate, poly vinyl alcohols, poly urethanes, poly carbonates, polystyrenes, including copolymers and other biocompatible polymer molecules.

Another means of preparing the near-IR fluorescing device is to incorporate the near-IR absorbing/emitting compound in one polymer and bind the polymer to the polymer of the graft tubing. That way a particular area could be caused to emit in the near-IR and not just the entire tubing or graft itself. In some embodiments, only the area that a needle is to be inserted will fluoresce. In another embodiment, each of the two insertion sites may fluoresce with a different near-IR wavelength (and may be displayed as different colors to the user).

For example, a patient in need of an AV graft may have a graft with a near-IR fluorescing material surgically implanted and positioned in an appropriate place, e.g., between an artery and a vein for access by a healthcare worker or technician. Once the graft is positioned in place in a patient, the healthcare worker may then view it using a near-IR imaging apparatus, such (e.g., see FIGS. 1B-1C and 9) and view the general area (e.g., an arm or leg) where the graft was placed and look for the appropriate near-IR florescence, which the apparatus may convert and display as a visible image. For example, a medical professional may then, while observing the images from the near-IR visualizing apparatus as it converts the near-IR emission into a visible image, insert the appropriate dialysis needles into the graft for use in dialysis of the patient.

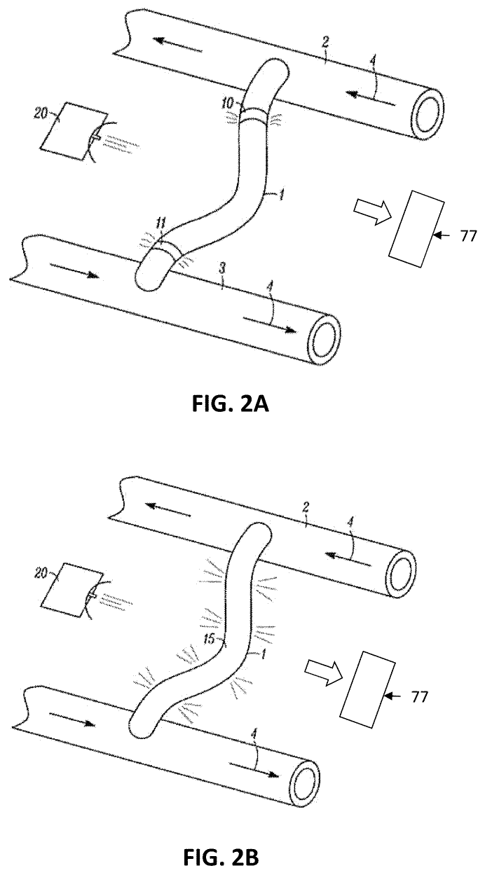

For example, FIG. 2A is a perspective view of an embodiment of a near-IR fluorescing device configure as a graft 1. The graft 1 is positioned between an artery 2 and a vein 3. Arrows 4 within the artery 2 and vein 3 indicate the direction of blood flow within that vessel. In this embodiment an artery needle insertion site 10 and a vein insertion site 151 are indicated as glowing bands. In this embodiment only the bands are made or coated with a near-IR fluorescing material and thus can be made to emit the same or a different emitted wavelength. The bands in this embodiment are depicted as glowing when the near-IR light source 20 is shined on the patient and imaged by an imaging apparatus 77 (e.g., a hand-held imaging reader). The near-IR light source 20 and reader 77 may be separate or they may be part of the same device (e.g., part of the hand-held imaging reader, as shown in FIGS. 1B-1C and 9). The bands could also be reinforced as needed since it is intended that there will be multiple needle sticks into this region of the graft.

FIG. 2B is a perspective view of another graft 1 configured as a near-IR fluorescing device. In this perspective graft 1 is made entirely of near-IR fluorescent polymer 15 such that upon exposure to the near-IR light source 20, the entire length of the graft 1 will glow. In this embodiment it would likely be that a single near-IR emitted wavelength would be impregnated into the polymer used for the graft. Other features known for other grafts could be included as well; however, the main feature of near-IR fluorescent polymer may remain the same.

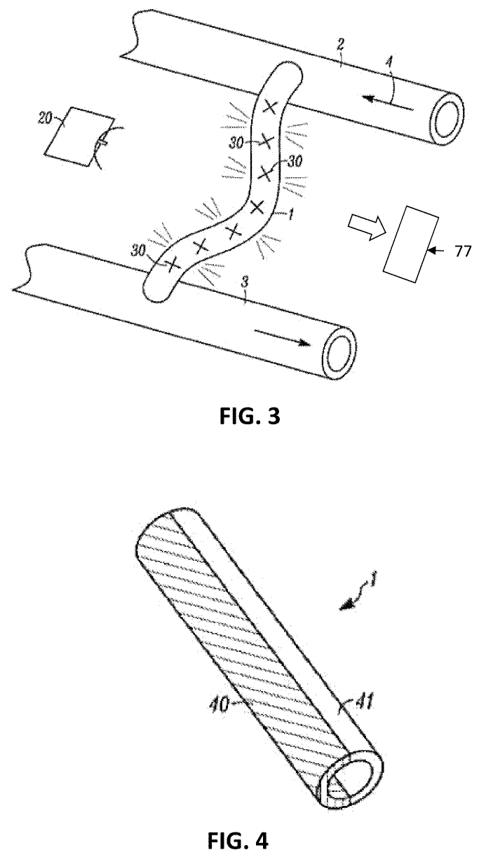

FIG. 3 shows an embodiment where near-IR fluorescing device is a graft 1 that has multiple stick sites indicated by X's 30. Each X 30 is made of a near-IR fluorescent material to indicate where to inject the needle but by giving them a distinctive shape they become easy to find. Clearly, the shape could be other than an “X”. For example, the shape could be a logo, alphanumeric characters, bull's eyes, or the like. The health care professional could rotate through each of the stick sites and then start again so as not to over stress any particular site by multiple injections.

FIG. 4 shows a different embodiment of a near-IR fluorescing device. In this embodiment the near-IR fluorescing device is a graft 1 made of two different near-IR fluorescent materials (e.g., polymers embedded, coated, impregnated, and/or cross-linked to different near-IR fluorescent materials). The front wall 41 is made of a first near-IR absorbing/emitting polymer and the back wall 40 is made of a second different near-IR emitting/absorbing material having different absorbing and/or emitting properties. This may help prevent the user from puncturing the back wall when passing a needle all the way through the graft.

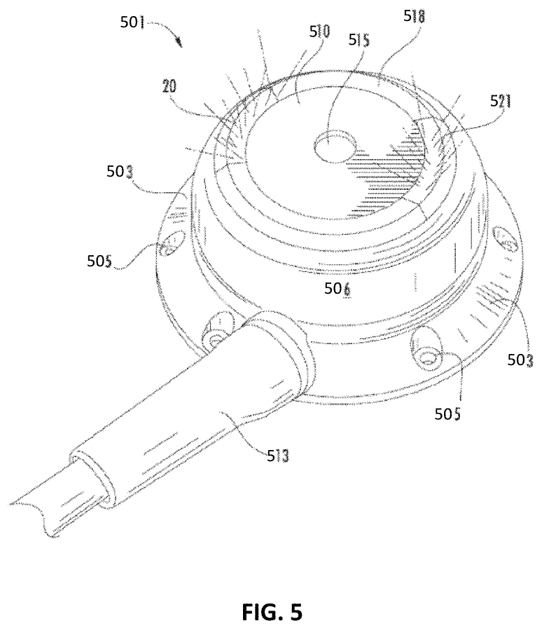

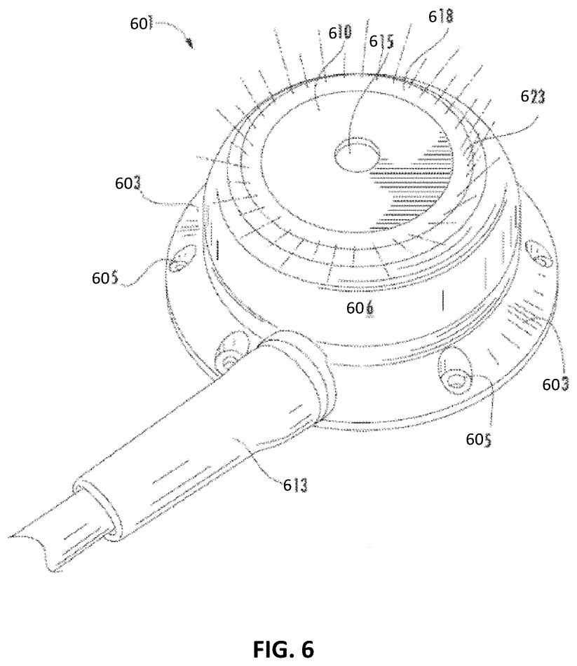

FIGS. 5 and 6 illustrate near-IR fluorescing device configured as ports. The apparatus can be totally or partially under the skin during use. It can be the portion closest to the skin or any portion as desired. It may be difficult to insert a needle or other device into a vascular access port without being able to visualize the port accurately. A “vascular access port” may be a biocompatible device which is placed subintimally and attached by sutures to the underlying fascia. They are designed for adding or taking away fluids to/from the vasculature where multiple access is required to the patient, for example, during chemotherapy treatment of cancer. A healthcare worker may use the port rather than continually inject or add new injection sites. The devices comprise an injection port for adding or taking a fluid away, a chamber and a tube which is in fluid communication with the chamber and a patient's vasculature. Placement of the device is where the access point is above or just under the skin making the port difficult to find by the healthcare worker. The vascular access port can be made of a biocompatible polymer or metal.

Medicaments, blood, nutrients or other material can be added or taken away from a patient's vasculature by inserting a needle into the port access hole and injecting or withdrawing fluid. Insertion of a needle through the skin and into the port should be as accurate as possible each time because poor needle insertion may cause infection and other complications.

Also described herein are near-IR fluorescing devices configured as ports that may incorporate a near-IR fluorescing material directly into the polymer or other matrix making up the port. The polymer can be injection molded or the like directly into the port shape from there. Examples of plastic which could incorporate the compound may include polyol(allyl car-bonate)-monomers, polyacrylated, polyethylenes, polypropylenes, polyvinyl chloride, polymethylmethacrylates, cellulose acetate, cellulose triacetate, cellulose acetate propionate, cellulose acetate butyrate, polyacetal resins, acetyl cellulose, poly vinyl acetate, poly vinyl alcohols, poly urethanes, poly carbonates, polystyrenes, including copolymers and other biocompatible polymer molecules.

While the near-IR fluorescing compound could be included in just a portion of the fabrication material, separate polymer-containing near-IR fluorescing materials (using the same or different materials) may be used. In one embodiment, only the area that around where a needle is to be inserted will fluoresce in the near-IR. In another embodiment two sites, e.g., one on each side of the injection site, could fluoresce in the near-IR.

A patient in need of a vascular access port may have a vascular access port surgically implanted and positioned in an appropriate place subintimally and sutured to the underlying fascia by a healthcare worker or technician. Once a vascular access port of the present invention is positioned in place in a patient, the healthcare worker would use a near-IR light source to illuminate the general area (e.g., arm, leg, etc.) where a vascular access port was placed and look for the appropriate fluorescence, using an imaging apparatus. The worker could then, while observing an image representing the near-IR fluorescence, insert the appropriate needles into the vascular access port. Where multiple vascular access ports are used, each site for needle insertion may fluoresce in the near-IR at a different wavelength, so that placement of each needle can easily be identified by use of separate (e.g. pseudo-colors representing the different wavelength). Either the absorption wavelength, the emission wavelength or both of the different near-IR materials may be used by the apparatus to indicate “different” regions.

FIG. 5 is a perspective view of an embodiment of a near-IR florescent vascular access port 501. The vascular access port 501 is positioned subintimally and fastened to the underlying fascia by suturing the port 501 by using suture holes 505 which are in base 503. In this view, the outer wall of chamber 506 has top 510. Top 510 has in the center access point 515 for insertion of a needle or the like. The outer ring 518 is the outer edge of top 510. In this view, left portion 520 and right portion 521 of ring 518 are made of near-IR fluorescing material. This is shown as one type of material but in other embodiments could be different near-IR fluorescing materials for 520 and 521 respectively. The port also has tubing 513 which extends to the vasculature as desired. The bands could also be reinforced as needed since it is intended that there will be multiple needle sticks into this region of the vascular access port.

FIG. 6 is a perspective view of another vascular access port 601 configured as a near-IR fluorescing device. In this example, a vascular access port 601 has the outer ring 618 made entirely of a polymer 623 including a near-IR fluorescing material such that upon exposure to the near-IR light of the appropriate exciting wavelength, the entire outer ring 618 will emit near-IR light that can be detected by a near-IR fluorescence readers and converted to a user-viewable visual display. In this embodiment a single near-IR absorbing/emitting material may be impregnated or coated into the port outer ring 618 used for the vascular access port. Other features known for other vascular access ports could be included as well.

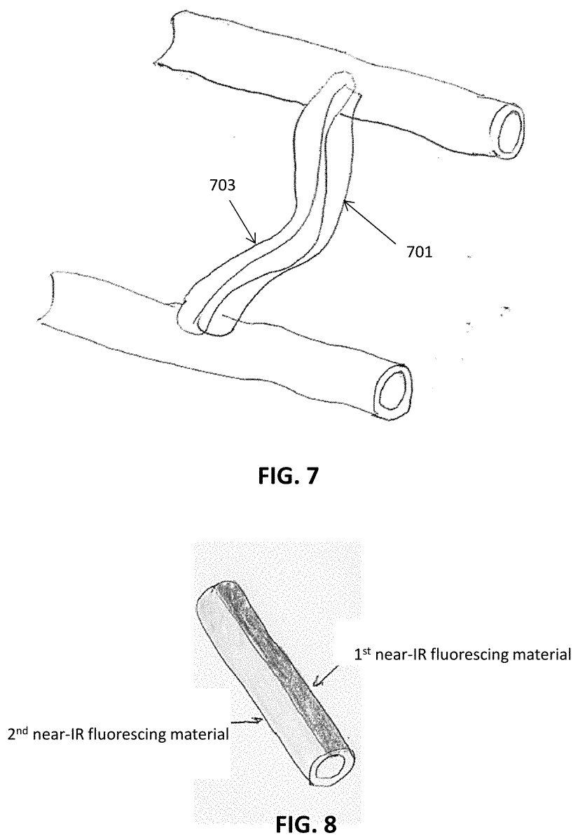

FIG. 7 illustrates another example of a graft having a pattern (e.g., stripes) of different near-IR absorbing/emitting materials. Two stripes, 703, 701, are shown, but other patterns (checkerboard, zig-zag, cross-hatch, etc.) may be used. FIG. 8 illustrates an example with different, adjacent, near-IR absorbing/emitting materials that may be used.

Although FIGS. 2A-8 illustrate ports and grafts, other exemplary devices may be used. For example, a cannulation device (e.g., angiocath, needle etc.), may be configured to be visible as a near-IR fluorescing device. Such a device may include, for example, an inner sharp metal hypodermic needle, an outer soft durometer slidable short catheter, and a coupling mechanism (e.g., a Luer lock) on the proximal side of the soft-durometer short catheter, and a fluorescent dye incorporated in the soft durometer catheter material, wherein the dye has photochemical characteristics that are compatible with the near-IR excitation and emission characteristics described herein. For example, the soft durometer material may be embedded with the same near-IR fluorescent material as the graft.

A method for guiding access to a near-IR fluorescing device configured as a graft may include: locating the fluorescent graft using a fluorescence imaging system (e.g., a hand-held imaging reader), positioning the image of the graft on the screen (or an augmented reality/virtual reality system) in a pre-determined location such that the needle guide track is attached to the outer enclosure of the imager guides the needle to the graft lumen from the correct point on the skin and at the correct angle to ensure that the graft lumen will be accessed. The needle may then be inserted into the guide track on the outer housing such that the needle starting point and angle are optimum to ensure that the needle impinges on the center of the graft. The needle may be inserted while watching for a “flash” (due to blood backing up into the needle body) to verify that the needle is in the graft lumen.

A method for guiding access to a near-IR fluorescing device configured as a graft lumen may include: locating the fluorescent graft using a fluorescence imaging system (e.g., an imaging reader), positioning the image of the graft on the screen (or visualizing using an augmented reality/virtual reality system) in a pre-determined location so that a needle guide track that is in, on or attached to the outer enclosure of the imaging reader can guide the needle to the graft lumen from a correct point on the skin and at the correct angle to ensure that the graft lumen will be accessed. A visualizable needle (e.g., including a near-IR absorbing/emitting material) may be inserted into the guide track so that the needle starting point and angle are optimum to ensure that the needle impinges on the center of the graft, and so that the needle trajectory can be imaged as it approaches the graft to verify correct trajectory. The needle may then be observed to identify the “flash” (e.g., when blood backs up into the needle body) to verify that the needle is in the graft lumen. Once in position, a slidable soft durometer outer sheath of the cannula may be pushed into the vessel and the sharp needle may be simultaneously retracted to leave the flexible catheter in the vessel.

As mentioned above, these apparatuses and methods may also be useful to ensure that the patency of a graft. For example, a method of monitoring the patency of a graft over time may include: accessing a near-IR fluorescing device configured as a graft (as above) or a feeder vessel to the graft, and recording an image of the graft without additional contrast enhancement. The graft may then be infused (or a region upstream of it may be infused) with a blood perfusion contrast agent whose photochemical characteristics are compatible with the excitation and emission characteristics of the imaging system (e.g., in the near-IR), and an image of the graft in the presence of luminal contrast agent may be recorded. The before and after images may be subtracted (e.g., by subtracting the non-contrast enhanced image from the contrast enhance imaged), to yield an image of the lumen of the graft. Later images may be compared to the image taken at time of implantation to gauge the build-up of intra-luminal plaque. For example, a series of images may be compared to assess the build-up of intra-luminal plaque over time to gauge when the graft might require cleaning in the future. A time-series of images may be used to gauge whether the graft is becoming leaky or damaged, thus indicating where not to stick in the future.

Also described herein are devices for reliably cleaning an occluded or partially occluded near-IR fluorescing device configured as a graft. For example, the device may include a catheter equipped with a lumen cleaning arm that is configured to recover the debris from cleaning. The catheter may have one or more radially viewing fibers that may be used to excite fluorescence from the graft wall and monitor the intensity of fluorescence returning from the wall to gauge the level of plaque build-up, and the nearness of the catheter to the wall thereby protecting the graft from accidental damage from the cleaning mechanism.

Thus, a method for safely and reliably cleaning an occluded or partially occluded near-IR fluorescing device configured as a graft may include inserting a distal embolic protection device into the lumen distal of the graft, inserting the catheter of (above) into the graft or into an adjacent feeder vessel, moving the cleaning catheter into the graft, monitoring the intensity of fluorescence using the radially viewing fibers, removing the intra-luminal plaque build-up on the graft by monitoring the intensity of fluorescence from the graft wall, and stopping the cleaning process prior to damaging the graft inner lumen by setting a threshold for fluorescence intensity at which it is believed that the lumen is substantially clear of occlusions but a small neo-intimal thickness of plaque remains to alleviate restenosis, and recovering the catheter and then the distal embolic protection device.

Also described herein are methods for locating/triangulating fiducial marks under the skin using near-IR fluorescence. For example, a method may include: implanting durable biocompatible markers containing a near-IR fluorescing dye, exciting and imaging the fiducial markers with an imaging device (such as those described above), building a 3D, 2D or quasi-3D representation of the region of the subdermal environment by registering the positions of the fiducial markers as the hand piece is moved around. The position of the probe may be monitored using a wireless signal from the probe, and this position may be interpreted by the controller/processor and used to build a picture in space of the probe position and orientation. The position of the probe may be monitored using one or more fiducial markers on the probe, and detected by the apparatus.

### PTFE Graft Example

In one example, Polytetrafluoroethylene (PTFE) grafts were coated in a near-IR absorbing and emitting material, in this example, indocyanine green (Exciton) and imaged with a commercially available near-IR fluorescence imaging device (e.g., Hamamatsu PDE NEO). The grafts were surgically implanted in a tunnel under the skin at representative depths by a vascular surgeon. Implant depths were determined using ultrasound.

Implanted grafts were readily detectable at about 5 mm depth, with some fluorescence detectable at deeper depths (e.g., up to 9 mm). This proof-of-principle experiment showed that visualization at depth is possible, and further, the intensity of fluorescence may be dependent on dye concentration, in the coated grafts examined. In particular, very high concentrations of coating were not as easily visible as less concentrated coatings. This may be due, at least in part, to self-absorbing or quenching the fluorescence. The optimum range of concentrations may be determined by coating and/or doping the graft host material at various concentrations and measuring the absorption and yield. The concentration range may be host (material) dependent as well as dye dependent. In some variations, there appears to be a relationship between material porosity and dye concentration. For example porosity in the material may concentrate the dye in the pores, locally increasing the concentration and therefore the confinement.

In general, it may be beneficial to use one or more near-IR absorbing and emitting material (“dye”) with very large absorption coefficient and a quantum efficiency close to 1, which may allow detection of deep grafts with reasonable optical excitation power at moderate dye levels.

Certain dyes (and/or classes of dyes) may resist degradation (e.g., light photobleaching, oxidation, recrystallization, changes in chemistry or morphology etc.), and may be sufficiently active post-implantation. For example indocyanine green (ICG) is an ionic dye, but PTFE is a relatively non-polar material. In some variations, a dye that is optically active in a non-polar form may be used with PTFE. When a polar host material or a biodegradeable material is used, dyes that are active in a polar (e.g., ionic—free charges) form may be used. For example, ICG, HITCI (e.g., 1,1′,3,3,3′,3′-Hexamethylindotricarbocyanine iodide), DTTCI (3,3′-Diethylthiatricarbocyanine iodide) etc. are examples of polar (ionic, Zwitterionic) dyes. Non-polar dyes, such as Langhals 5b may be used with a non-polar host. Rylenes like (5b) may have very high quantum yield (e.g., close to one), very high photostability (e.g., can repeatedly absorb and re-emit photons without changing chemically), and very high resistance to atmospheric oxidation. Indocyanine green typically has a quantum efficiency of around 20% and a finite photobleaching lifetime. Thus, in some variations, ICG (and similar dyes) may be used for biodegradeable implants, particularly those having polar host materials. In some variations, rylenes may be used with non-polar (e.g., PTFE) host materials.

In some example, the amount or concentration of the near-IR fluorescent material may within a target range (e.g., an upper concentration and a lower concentration). In some variations, concentrations about the upper concentration may result in quenching, decreasing the fluorescence. For example, in one variation, the fluorophore (the near-IR fluorescing material) is ICG or a related dye, and the concentration may be about 0.5 milligrams per kg of base material (e.g., the base material may be the graft material, when the medical device is a graft). Below a lower limit is the fluorescence may not be detectable (typically greater than zero molecules because the dye must emit above a noise floor to be detected). For example, the range of concentration may be between about 0.03 to about 0.95 micromolar (e.g., between about 0.03 to about 0.075 micromolar, between about 0.05-0.65 micromolar, etc.). For example, the range may be between 0.000001% by weight and about 0.001% by weight (e.g., between 0.00001% by weight and 0.0001% by weight, etc.). In some variations, the concentration may be between about 0.01 mg/kg in polymer to about 0.75 mg/kg in polymer (e.g., between about 0.02 mg/kg and about 0.60 mg/kg in polymer/base, between about 0.03 mg/kg and about 0.055 mg/kg in polymer/base, between about 0.04 mg/kg and about 0.5 mg/kg in polymer/base, etc.) The range and the optimum/midpoint may vary with the different dyes, and with the porosity of the implant material.

In some variations, dyes that are sensitive to oxidation or recrystallization may be powder coated onto the grafts and protected with an overlayer of, e.g., a clear, non-permeable material to isolate them from atmospheric oxygen pre-implantation. Coated implants may be stored in a light-proof material like foil. The protective layer could be biodegradeable, which may allow the dye to be activated by interacting with a polar fluid environment such as that found in a live person.

In this example, the fluorescence of the donor material did not appear to depend on the skin pigmentation. For example, skin having significant melanin in the epidermal/dermal layer did not impact visualization of SWIR fluorescence. The use of SWIR imaging described herein may be effectively used with any skin type. In addition, the visualization may be modified and/or improved by the use of any of the imaging/fluorescence imaging apparatuses described herein.

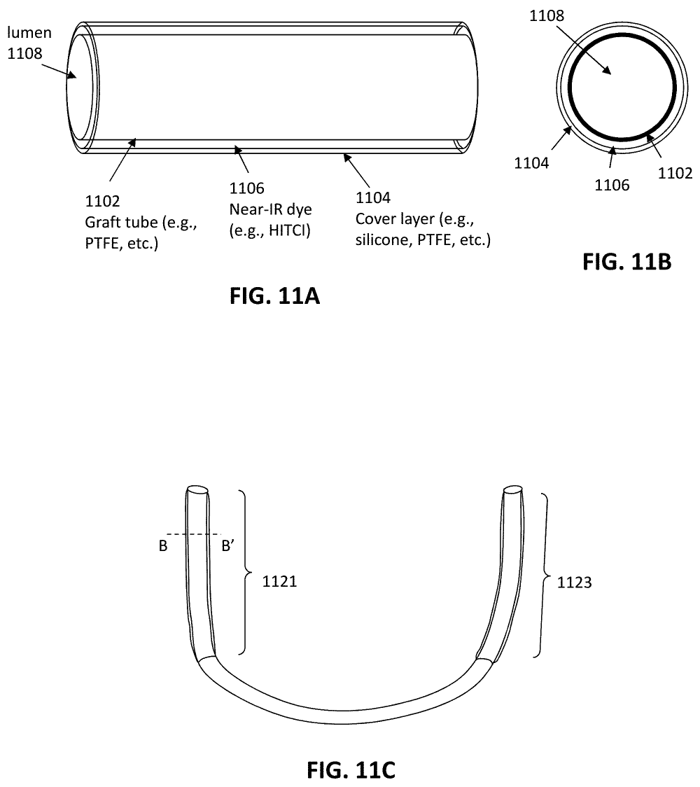

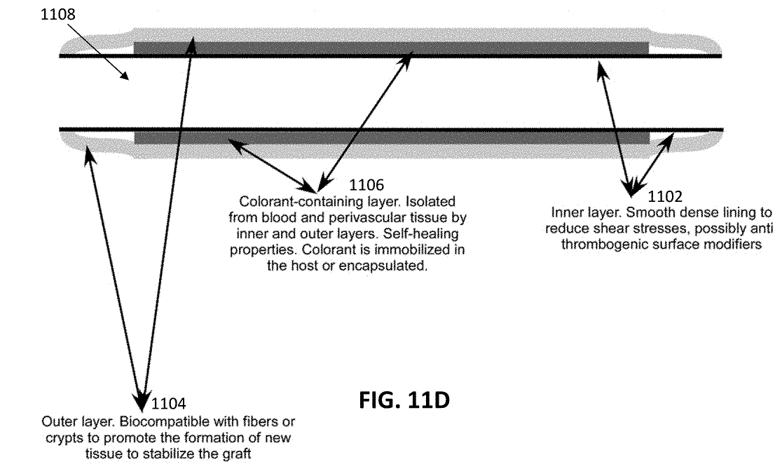

FIGS. 11A-11D illustrate another variation of an implant, configured as an AV shunt in this example, that may include a near-IR dye for visualizing through the patient's skin. In some variations the dye may be part of a marking medium (e.g., may be combined and/or encapsulated with a substrate, such as silicone, and applied as a marking medium to an implant or insertable device to allow visualization, in real-time, even when positioned within the tissue.

For example, in FIG. 11A a region of an AV shunt or graft is shown, including three separately layers. The inner layer 1002 is typically a smooth, inert, biocompatible lining that may be formed of a smooth and dense material to prevent or limit shear stress; this layer may be or may include an antithrombegenic material. For example, in some variations the outer layer is polytetrafluoroethylene (e.g., PTFE, ePTFE, etc.). The inner layer may provide structural support (e.g., stiffness) to the implant, or may be included with one or more additional layers. The near-IR dye (e.g., included as part of a marking medium) may then be added atop 1106 the inner layer. The near-IR dye may be added to a portion of the device, including in a pattern, as described above. For example, the pattern may be a spiral/helical pattern. In some variation, the pattern includes one or more bands, strips, etc. As will be described in greater detail below, only some regions (e.g., needle stick regions, such as the venous and/or arterial regions of the implant) are labeled with the near-IR dye.

For example, in FIG. 11A, the near-IR dye is applied as a layer 1106 over the inner layer 1102. This colorant layer may be isolated from the patient's blood and perivascular tissue by the inner layer and by a separate outer layer 1104, preventing release of a substantial amount of the near-IR dye material into the subject's body. In some variations the near-IR dye is included within a substrate having neutral optical properties within the near-IR absorbing and receiving range of the dye (e.g., HITCI) used. As mentioned the substrate may be a self-healing material such as silicone. To prevent the dye material from releasing into the patient's blood or tissue, the implant may, in some variations, include an outer layer 1104 covering the middle near-IR absorbing/emitting layer. The covering layer may be a biocompatible material. In some variations the outer covering material is configured to promote tissue in-growth. For example, the outer covering material may include fibers or crypts to promote the formation of new tissue that may help stabilize the graft. In some variations the outer covering layer may be PTFE or other polymeric material. The material may be transparent within the near-IR range.

In some variations the near-IR dye material is encapsulated in particles (e.g., microparticles, nanoparticles, beads, etc.) that may be enclosed between an inner and outer layer, as shown in FIGS. 11A-11B, or they may be coated directly onto the outer surface of the implant. The encapsulating substrate may be a polymeric material, including silicone (e.g., one or more polysiloxane).

FIG. 11B shows a section through the implant of FIG. 11A, showing the inner layer 1102 forming the body of the implant (e.g., such as an AV shunt), a middle near-IR layer (e.g., marking medium) and an outer covering layer 1104. In some variations both the inner and outer layers are PTFE, while the marking medium includes a near-IR dye (e.g., an organic laser dye).

FIG. 11C illustrates one example of an AV graft including a three-layered, near-IR labeled region similar to that shown in FIGS. 11A-11B. In FIG. 11C, two regions are labeled and may be visualized. The first region 1121 may be a venous region that can be connected to the venous side of the fistula; the second region 1123 may be an arterial region that can be connected to the arterial side of the fistula. Having separately labeled arterial and venous regions may allow better accuracy when visualizing and determine where to stick needles into the AV shunt, e.g., during dialysis.

FIG. 11D is an exemplary section through a three-layered, near-IR labeled region of an implant such as an AV shunt having a central lumen 1108. The inner layer 1106 may include a colorant (e.g., near IR dye) containing layer. This middle or inner layer 1106 may include, for example, between 0.005% and 0.1% HITCI. An outer layer 1104 may be applied over the inner and middle layer to secure the dye-containing layer away from the body.

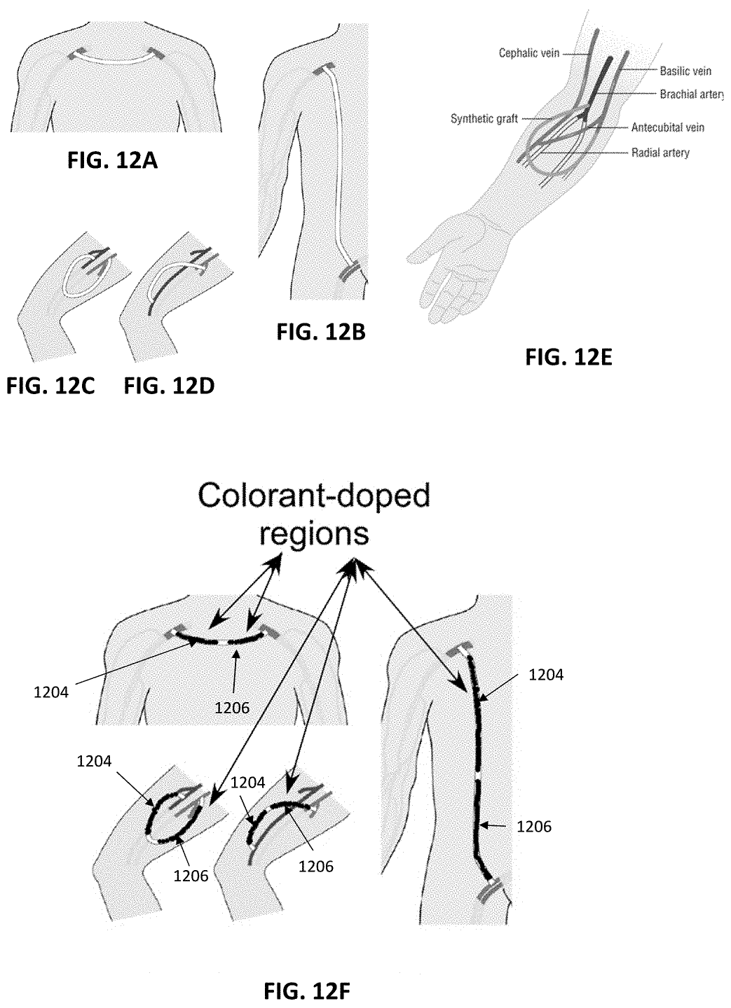

In FIG. 11D the dye layer is tapered or terminated towards the ends so that the sutures don't create a path through the graft for the dye to wick out. The break in the colorant region to denote the arterial and venous stick regions could happen in a similar manner. In this example (also shown in FIG. 12F, below) the dye is not positioned either near the suture points or the middle of the graft to denote a break point and/or avoid the curve on a looped graft (e.g., to mitigate kinking).

FIGS. 12A-12E illustrate various AV shunts and shunt locations that may be used. For example, FIG. 12A shows a chest shunt between subclavian artery and vein. FIG. 12B shows a shunt from subclavian to groin region. FIGS. 12C and 12D show shunts in the upper leg. FIG. 12E shows a shunt in the patient's arm (e.g., between the brachial artery and the antecubital vein). The AV shunts described herein may be used in any appropriate region of the body.

In general, it may be beneficial to mark (via near-IR marking) only regions of the implant that need to be imaged, for example, for later access. For example, an AV shunt may be marked in the regions into which one or more needles will be inserted. This may beneficially allow the avoidance of regions into which the needle should not be inserted, including bent regions, or other regions that may be damaged (e.g., may crimp, kink, rupture, etc.). FIG. 12F shows the same exemplary shunts from FIGS. 12A-12D, further indicating only two regions, an arterial region 1204 and a venous region 1206. The regions not include the dye (e.g., regions which may be used to suture the device within the body, etc.) may therefore be avoided when using the apparatuses described herein to image and/or insert a needle into the shunt, e.g., for dialysis.

As discussed above the near-IR dye may be incorporated or encapsulated into a substrate, such as silicone, that is self-healing following one or more needle punctures. In addition, the inner and/or outer layers may be self-healing, which may prevent leakage of the dye from the labeled regions. For example, a near-IR dye (e.g., HITCI) may be integrated into a silicone layer between two PTFE layer(s). Alternatively or additionally, the dye may be tethered to nanoparticles/microparticles that may be individually encapsulated to prevent or limit release of the dye, even when the region including the dye is penetrated by one or more needles.

Although FIG. 12F shows the exemplary shunt in which the entire arterial and venous regions are separately marked by the dye, as described above, one or more marking patterns may be used. For example, a spiral marking pattern may be used (e.g., helically traversing the arterial and/or venous regions), a checkered pattern, etc. In some variations, less than x % of the target marked region is marked (e.g. less than 70%, less than 60%, less than 50%, less than 40%, less than 30%, etc. of the venous and/or arterial regions is marked). This may reduce the likelihood of the needle penetrating through the dye and/or removing or leaking dye into the body. By distributing the marking around the overall region to be marked (e.g., the venous and/or arterial regions of an AV stent), the entire region may be imaged, particularly (but not necessarily) in variations in which the apparatus processes the near-IR image before displaying it, as described in further detail below. For example, the apparatus may process an image to define an outline of the putative marked regions (e.g., arterial and/or venous regions of the AV shunt) by edge detection, regardless of the uniformity of the marking and/or image received, which may help correct for both non-uniformity and/or low intensity images.

Thus, any of the systems described herein may be adapted or configured to illuminate a portion of a patient's body, such as the skin of an arm, leg, chest, etc., with near-IR illumination (e.g., near-IR illumination that is <800 nm, e.g., between 700-800 nm in some, non-limiting, variations), and to receive near-IR images back, e.g., by receiving, and in some cases filtering selectively for, near-IR wavelengths above or below the illumination wavelength range (e.g., 800 nm or greater).

The received image may be processed in real time, enhance the received image(s) prior to display. For example, the received near-IR images may be enhanced to determine one or more edges likely to correspond to the actual edges of the labeled implant. For example, when imaging a near-IR labeled implant (e.g., graft) through the skin, the image may appear blurry, and the edges may actually extend beyond the true edges of the implant, due in part to the optical properties of the tissue through which the implant is being imaged. The image intensity may also affect the apparent and misleading spreading of the imaged edges. Because this may negatively impact the accuracy when using this imaging to guide a needle stick, the apparatuses (e.g., systems) described herein may be configured to correct for this. In some variations the imaging apparatus may include processor for processing the images, in real time or near real time, to detect edges of the implant and adjust for expansion of the edges. Edge detection may be performed in any appropriate manner (e.g., by Gaussian blur edge detection, Canny edge detection, differential edge detection, etc.). The edges may be contracted (e.g., by a fixed percentage or by a percentage based on the nearby intensity and/or average intensity of the marked region) inward, e.g., towards the marked region, in order to account for erroneous spreading of the image edges. If a fixed percentage is used, the fixed percentage may be between 2% and 50% (e.g., 5%, 10%, 15%, 20%, 25%, etc.).

As mentioned the dye may be patterned (e.g., in a spiral-wrap or some other structured pattern), resulting in alternating bright and dim fluorescence. The apparatus may be configured (e.g., the edge detect engine associated with the apparatus) to detect the outer edges from the dye patterns corresponding to the actual edge(s). Also since the dimmer regions are fluorescence from the bottom of the graft, any internal blockages, atherosclerosis formation, clot formation may cause these regions to dim further. In some variations the apparatus may be configured to detect the onset of graft failure due to internal blockage based on the alternating bright and dark sections.

Alternatively or additionally, the image may be processed to track one or more prior needle penetrations, and prior penetration sites may be marked. For example, in some variations, the system may track a needle penetration into the implant (e.g., an AV shunt) when using a marked needle and storing the location of needle penetrating. The apparatus may therefore include a memory that may be updated with prior needle injection sites. Alternatively or additionally, in some variations the apparatus may examine the image for regions having near-IR image markings that are characteristic of needle penetrations, such characteristic patterns (dots, slits, etc.) in which near-IR intensity is different from the surrounding (more uniform) intensity. These patterns may be approximately the same size as the needle penetration size.

In any variation of the apparatuses described herein, the system may determine and suggest or display one or more proposed needle stick sites on the graft. Proposed needle penetration (needle stick) sites may be determined based on the intensity of the image (e.g., upper regions may tend to emit more brightly), and/or based on the prior needle penetration locations, as mentioned above. Proposed needle penetration locations may be displayed directly on the image(s) of the graft and may be marked in a different color or pattern (e.g., in red, green, etc. with blinking lights, etc.); proposed targets for needle penetration may be shown as cross-hair regions or as bullseye-type targets, etc. One or more proposed needle-penetration location(s) may be illuminated directly, in real time, on the patient's skin (e.g., by an illuminated indicator projected on to the patient's skin); in some variations this may cause one or more marks to appear on the skin in the needle-penetration location(s). In some variations a light-sensitive (photosensitive) solution may be applied to the patient's skin and the light marking may result in a physical mark (e.g., dye mark) being made on the patient's skin over the proposed location by reaction with the photosensitive solution. Photosensitive solutions may be applied wet and may dry; illuminating them with a particular wavelength of light may result in a color change on the skin to which they were applied.

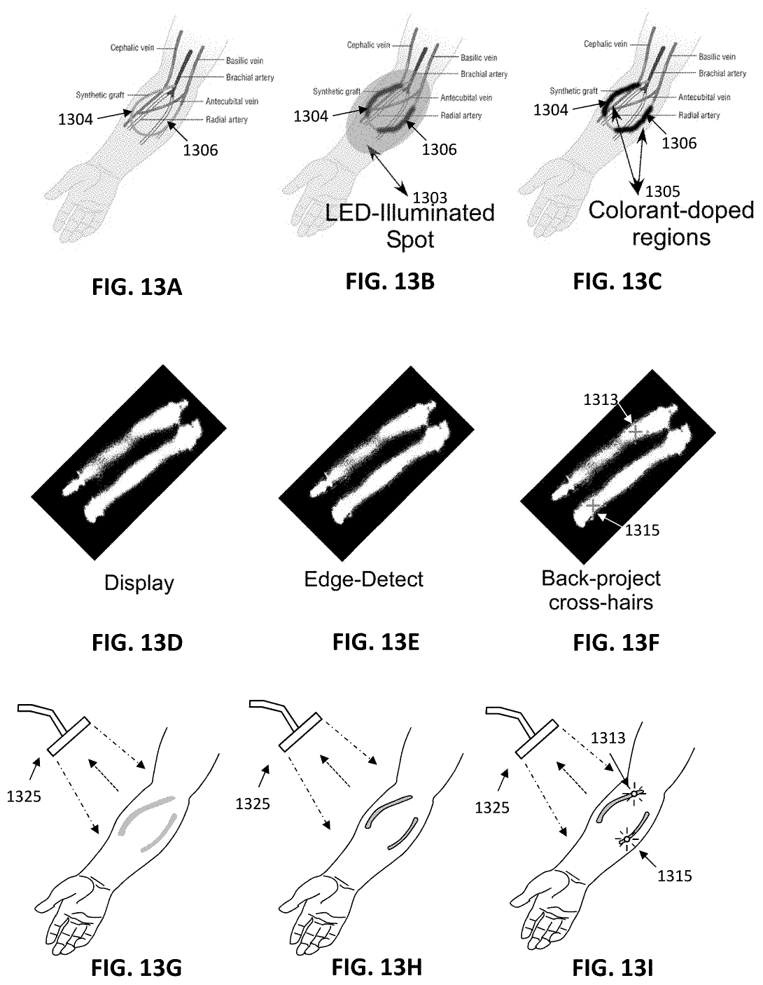

FIGS. 13A-13C illustrate examples of imaging an implant marked with a pattern of near-IR dye through the skin. In FIGS. 13A-13C an AV shunt that is labeled in two regions, an arterial region 1304 and venous region 1306, is implanted into the patient's skin, as shown in FIG. 13A. Near-IR illumination (“LED illumination spot” 1303) may be applied to the skin by a detection apparatus such as those described herein, as shown in FIG. 13B. In FIG. 13C, near-IR light is emitted by the dye on the two regions of the graft, and this light may be detected through the intact skin, as shown by FIG. 13C. The marked (“colorant doped regions” 1304, 1306) may be displayed to the practitioner (e.g., doctor, technician, nurse, dialysis technician, etc.).

FIGS. 13D-13F illustrate example of images that may be displayed. As described above, in FIG. 13D an image of the near-IR emission from the implant (AV graft) is shown. The display may be on a monitor near the patient (e.g., next to above, adjacent, etc.) so that the practitioner may use it for guidance in performing the needle stick(s). Alternatively or additionally, the display may be projected directly onto the patient's skin. Alternatively or additionally, a virtual reality/augmented reality may be used to show the output of the apparatus.

The output may be in real time. In some variations the output is an image of the florescence/emission of the near-IR signal, as shown in FIG. 13D. In some variations the output may include a visible light image onto which the near-IR image is superimposed. In any of these variations, as described above and shown in FIG. 13E, the apparatus may detect edges from the near-IR image captured, and/or may adjust the borders/size of the near-IR image to more accurately reflect the size of the implant beneath the skin. In FIG. 13E edge detection may be used to indicate the outline of the implant even when parts of the image are lower in intensity. FIG. 13F illustrates an example in which proposed needle insertion sites are provided by the apparatus. In FIG. 13F two needle insertion sites 1313, 1315 (e.g., an arterial and a venous insertion site) are shown as cross-hairs on an image of the labeled regions of the AV shunt.

FIGS. 13G-13I illustrate examples of projections (real-time projections) of the near-IR images of the shunt directly onto the patient's skin. In FIG. 13G the apparatus 1325 projects excitation near-IR light onto the patient's skin and detects emitted near-IR light from a labeled implant beneath the skin; the apparatus may also project an image onto the patient's skin showing the location of the implant beneath the skin. The image may be processed, as described above, e.g., to detect edges (as shown in FIG. 13H), adjust the edge location/size, and/or to suggest one or more needle insertion sites, as shown in FIG. 13I. In some variations, only the proposed needle insertion sites are shown.

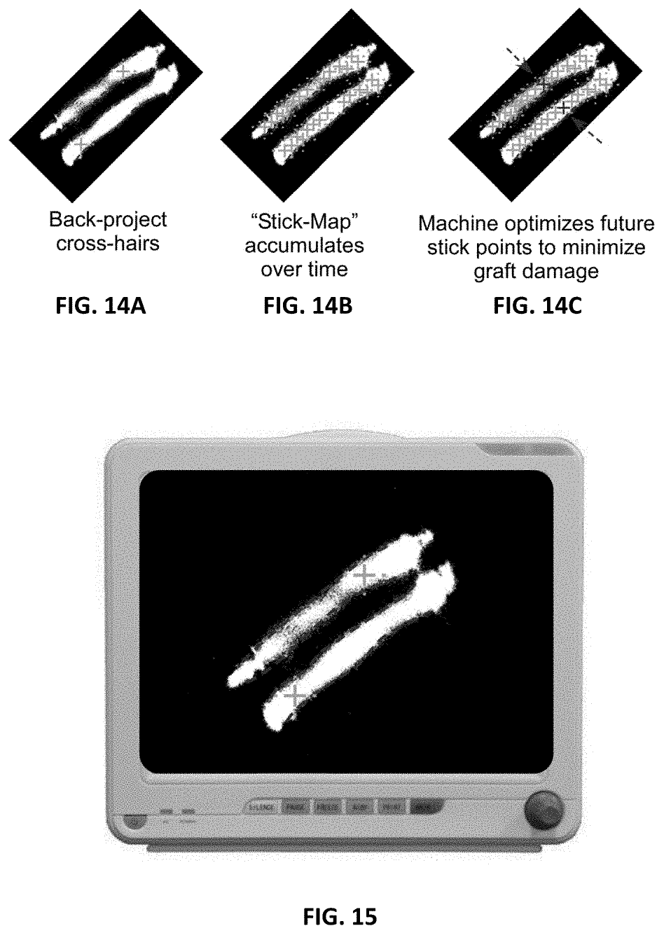

FIGS. 14A-14 illustrate another example in which prior needle insertion sites are illustrated. In FIG. 14A the apparatus shows just two prior insertions sites, as described above, shown here as an “x” for each prior insertion site. In FIG. 14B, multiple prior insertion sites are shown labeled. In FIG. 14C the apparatus has proposed/suggested two new needle insertion sites, which may be displayed in a different color. As mentioned, the display may be on a screen (e.g., FIG. 15), an augmented reality display, and/or projected onto the patient's skin.

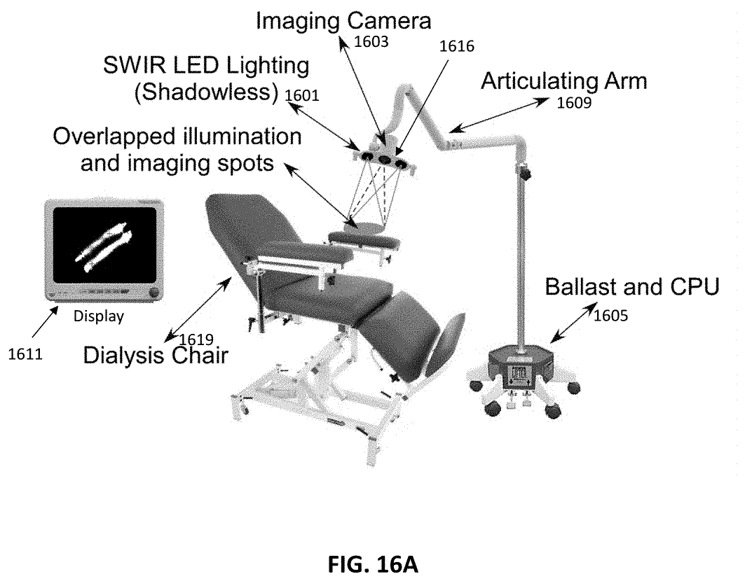

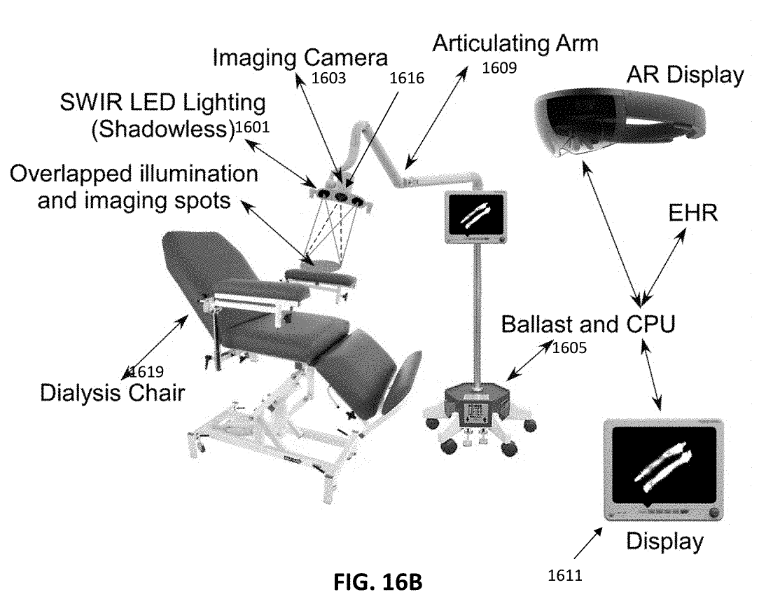

FIG. 16A is another example of an apparatus for imaging an implant labeled with a near-IR dye. In FIG. 16A the apparatus may include a near-IR illumination source 1601 (e.g., SWIR LEDs), an imaging near-IR camera 1603 (Imaging camera), a processor 1605, and a memory. Light and camera may be mounted on a positionable arm 1609. The processor may be configured to process the image(s) as described above. Optionally, a display 1611 may be used to display the image and/or the processed image may be displayed back onto the skin of the patient via a projector 1616 that may be combined with the illumination source and/or camera. In some variations, the apparatus may include and/or may be mounted to a chair 1619, table or bed.

FIG. 16B is another example of an apparatus for imaging an implant that is labeled with a near-IR dye. In FIG. 16B the apparatus may also be configured for use with an adaptive reality subsystem that may include an AR display 1632 (e.g., googles). The apparatus processor 1605 (e.g., CPU) may be configured to display to a monitor and/or an adaptive reality output.

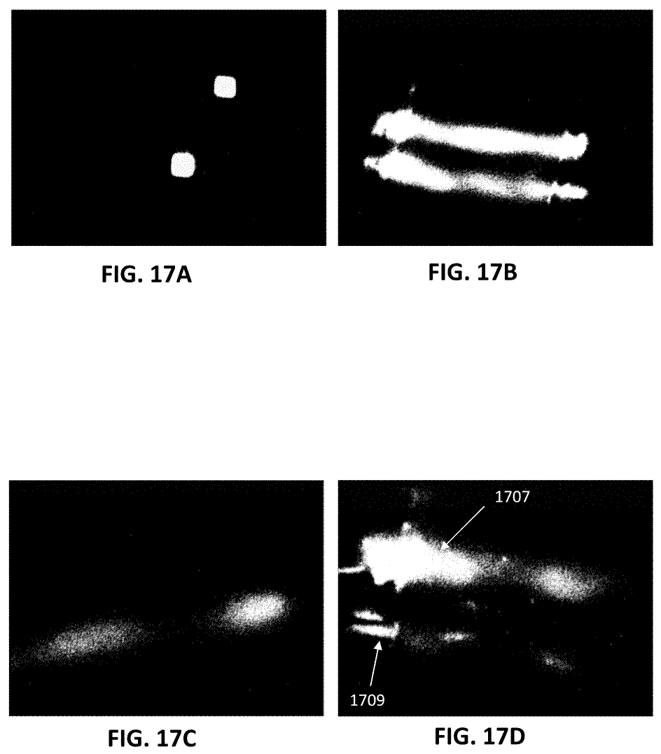

FIGS. 17A-17D illustrate examples of images that may be taken with a system similar to those shown in FIGS. 16A-16B. FIG. 16A is a calibration image taken with a control device having two square regions marked with a fluorescent dye responsive to the excitation and emission properties of the imager. Prior to encapsulation of the dye, an implant, such as an AV graft, that is coated with a near-IR dye may leave some of the dye behind in the tissue. This is illustrated in FIG. 17B; in this example an implant (an AV graft) coated with HITCI (e.g., 0.1%) was inserted into the tissue (e.g., a Caucasian male) as part of a preliminary cadaveric study. In this example, a tunnel into the tissue (approximately 2-4 mm deep) was formed and the graft inserted, then removed. The residual near-IR dye left behind was imaged, as shown in FIG. 17B. Similarly, a graft coated with 0.015% HITCI also left behind a substantial residue. To prevent leaching/transfer of the dye into the patient, encapsulated/sealed or layered implants, such as those shown above in FIGS. 11A-11D were examined, as shown in FIGS. 17C and 17D. These implants included an inner layer of PTFE, a silicone-dye layer inner layer (silicone with either 0.015% w/w or 0.1% w/w of HITCI) and an outer layer of PTFE. Implantation of an AV shunt labeled with 0.015% HITCI was imaged through approximately 4 mm of tissue, as shown in FIG. 17C. LEDs emitting light at about 750 (with around 30 nm bandwidth) were used to illuminate the region of the body including the implant. A camera having a long-pass filter (with a cutoff of around 800 nm) was used for imaging. FIG. 17D shows another example of a pair of grafts inserted 2-4 mm into the tissue and imaged as described above. In FIG. 17D, the top implant 1707 was labeled with approximately 0.1% w/w HITCI dye. The lower implant 1709 was labeled with 0.015% w/w HITCI dye. The image shown is auto-scaled to the highest intensity point (the point shown on the top implant).

Any of the methods (including user interfaces) described herein may be implemented as software, hardware or firmware, and may be described as a non-transitory computer-readable storage medium storing a set of instructions capable of being executed by a processor (e.g., computer, tablet, smartphone, etc.), that when executed by the processor causes the processor to control perform any of the steps, including but not limited to: displaying, communicating with the user, analyzing, modifying parameters (including timing, frequency, intensity, etc.), determining, alerting, or the like.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

In general, any of the apparatuses and methods described herein should be understood to be inclusive, but all or a sub-set of the components and/or steps may alternatively be exclusive, and may be expressed as “consisting of” or alternatively “consisting essentially of” the various components, steps, sub-components or sub-steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

1. An arteriovenous shunt (AV shunt) implant device, the device comprising: an elongated tubular body the body having an inner lumen formed of an inner layer; a first middle layer extending at least partially over the inner layer, the first middle layer comprising a first substrate and a near-infrared (near-IR) dye that absorbs and emits in the near IR wavelength range; and a first outer layer extending over the first middle layer and sealing the first middle layer between the first outer layer and the inner layer, wherein the near-IR dye is at a concentration of between 0.001% to 0.5% w/w and comprises one or more of: 1,1′,3,3,3′,3′-Hexamethylindotricarbocvanine iodide (HITCI), and a rylene dye.

2. (canceled)

3. The device of claim 1, wherein the near-IR dye is at a concentration of between 0.001% w/w and 0.1% w/w.

4. The device of claim 1, wherein the tubular body comprises a second middle layer separate from the first middle layer, wherein the second middle layer comprises a second substrate and a second near-IR dye.

5. The device of claim 4, wherein the second middle layer is covered by the first outer layer or a second outer layer extending over the second middle layer and sealing the second middle layer between the second outer layer and the inner layer.

6. The device of claim 4, wherein the second near-IR dye is the same as the first near-IR dye and the second substrate is the same as the first substrate.

7. The device of claim 1, wherein the first substrate comprises silicone.

8. The device of claim 1, wherein the inner layer comprises polytetrafluoroethylene.

9. The device of claim 1, wherein the first outer layer comprises a biocompatible material that is greater than 50% transparent to light between about 700-850 nm.

10. The device of claim 1, wherein the first outer layer comprises polytetrafluoroethylene.

11. The device of claim 1, wherein the near-IR dye extends in a pattern over the inner layer.

12. An arteriovenous shunt (AV shunt) implant device, the device comprising: an elongated tubular body the body having an inner lumen formed of an inner layer; an arterial region comprising a first middle layer surrounding and extending partially along a first length of the inner layer, the first middle layer comprising a first substrate and a first near-infrared (near-IR) dye that absorbs and emits in the near IR wavelength range; and a first outer layer extending over the first middle layer and sealing the first middle layer between the first outer layer and the inner layer; and a venous region comprising a second middle layer surrounding and extending partially along a second length of the inner layer, the second middle layer comprising a second substrate and a second near-infrared (near-IR) dye that absorbs and emits in the near IR wavelength range; and wherein the second middle layer is covered by the first outer layer or a second outer layer, further wherein the first and second near-IR dyes is at a concentration of between 0.001% to 0.5% w/w and comprises one or more of: 1,1′,3,3,3′,3′-Hexamethylindotricarbocyanine iodide (HITCI), and a rylene dye.

13. (canceled)

14. The device of claim 12, wherein the first and second near-IR dyes are at a concentration of between 0.001% w/w and 0.1% w/w.

15. The device of claim 12, wherein the first and second near-IR dyes are different near-IR dyes.

16. The device of claim 12, wherein the second middle layer is covered by the first outer layer so that the first outer layer seals the second middle layer between the first outer layer and the inner layer.

17. The device of claim 12, wherein the first substrate and the second substrate comprises silicone.

18. The device of claim 12, wherein the inner layer comprises polytetrafluoroethylene.

19. The device of claim 12, wherein the first outer layer comprises a biocompatible material that is greater than 50% transparent to light between about 700-850 nm.

20. The device of claim 12, wherein the first outer layer comprises polytetrafluoroethylene.

21. The device of claim 12, wherein the first near-IR dye extends in a pattern over the inner layer.

22.-26. (canceled)
